(19) United States
(12) Patent Application Publication
(10) Pub. No.: US 2018/0245102 A1
(43) Pub. Date: Aug. 30, 2018

(54) METHODS OF MICROBIALLY PRODUCING ACIDS AND MINERALS AND USES THEREOF

(71) Applicant: ELEMENTAL ORGANICS, LLC, Sandy, UT (US)

(72) Inventors: Kenneth R. LLOYD, Bishop, CA (US); Jon BAKER, Salt Lake City, UT (US); John Jay LUND, Sandy, UT (US)

(73) Assignee: ELEMENTAL ORGANICS, LLC, Sandy, UT (US)

(21) Appl. No.: 15/757,643

(22) PCT Filed: Sep. 2, 2016
(86) PCT No.: PCT/US2016/050253

§ 371 Date: Mar. 5, 2018

Related U.S. Application Data
(60) Provisional application No. 62/278,831, filed on Jan. 14, 2016.
(60) Provisional application No. 62/214,656, filed on Sep. 4, 2015.

(51) Int. Cl. C12P 3/00; C05D 9/02; C05C 3/00; C05B 17/00; C05B 1/02; C05C 5/00; C05C 1/00; C01B 17/54; C01B 17/69

(52) U.S. Cl. CPC C12P 3/00; C05D 9/02; C05C 3/00; C05B 17/00; C01B 17/69; C05C 5/00; C05C 1/00; C01B 17/54; C05B 1/02

(57) ABSTRACT

A method of producing sulfuric acid can include: obtaining a microbial culture that produces sulfuric acid; placing the microbial culture in a bioreactor in an aqueous environment; introducing an aqueous, liquid or gaseous sulfur supply into the bioreactor; and culturing the microbial culture with the sulfur supply sufficiently so that sulfuric acid is produced. The sulfur supply can be from aqueous or gaseous sulfur dioxide and/or dihydrogen sulfide, or sulfurous acid. The microbes are any microbes that processes sulfur, such as natural microbes that processes sulfur, genetically modified microbes that processes sulfur, cultivated microbes en.) that processes sulfur, purchased microbes that processes sulfur. The microbes may also include other type of microbes that facilitate culturing of the sulfuric acid producing microbes. The sulfuric acid can be produced at ambient conditions. In one aspect, the process is a batch process or a continuous process.

### CROSS-REFERENCE

This patent application claims priority to U.S. Provisional Application No. 62/278,831 filed Jan. 14, 2016 and U.S. Provisional Application No. 62/214,656 filed Sep. 4, 2015, which provisional applications are each incorporated herein by specific reference in their entirety.

### BACKGROUND

Acids and mineral salts have significant industrial applications. However, obtaining acids and mining for mineral salts can be complicated and expensive endeavors. As such, synthetic preparation of acids and mineral salts can be beneficial.

### SUMMARY

In one embodiment, a method of producing sulfuric acid can include: obtaining a microbial culture that produces sulfuric acid; placing the microbial culture in a bioreactor in an aqueous environment; introducing an aqueous, liquid or gaseous sulfur supply into the bioreactor; and culturing the microbial culture with the sulfur supply sufficiently so that sulfuric acid is produced. In one aspect, the sulfur supply is from gaseous sulfur dioxide and/or dihydrogen sulfide. In one aspect, the sulfur supply is aqueous sulfur dioxide and/or dihydrogen sulfide. In one aspect, the sulfur supply is sulfurous acid. In one aspect, the produced sulfuric acid is MPSA from an organic sulfur supply. In one aspect, the microbes are any microbes that processes sulfur. In one aspect, the microbes are any natural microbes that processes sulfur. In one aspect, the microbes are any genetically modified microbes that processes sulfur. In one aspect, the microbes are any cultivated microbes that processes sulfur. In one aspect, the microbes are any purchased microbes that processes sulfur. In one aspect, the microbes include other type of microbes that facilitate culturing of the sulfuric acid producing microbes. In one aspect, the sulfuric acid is produced at ambient conditions. In one aspect, the process is a batch process. In one aspect, the process is a continuous process.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an embodiment of a sulfurous acid producing system, which can be modified to include a bioreactor having microbes that produce acids and/or minerals.

FIG. 2 illustrates an embodiment of a sulfurous acid and sulfuric acid producing system includes a bioreactor having microbes that produce sulfuric acid and/or sulfate minerals.

FIG. 3 illustrates an embodiment of a bioreactor for producing sulfuric acid.

FIG. 4 illustrates an embodiment of a bioreactor for producing ammonium sulfate.

FIG. 4A illustrates an embodiment of a bioreactor for producing phosphate salts.

FIG. 4B illustrates an embodiment of a bioreactor for producing mineral salt.

FIG. 5 is a side cross-sectional view that illustrates an embodiment of a compositing silo.

FIG. 5A is a top cross-sectional view that illustrates an embodiment of an auger system in the composting silo of FIG. 5.

FIG. 6 illustrates an embodiment of a bioreactor for producing nitrite.

FIG. 7 illustrates an embodiment of a bioreactor for producing nitrate.

FIG. 8 illustrates an embodiment of a bioreactor for producing nitrite and/or nitrate.

FIG. 9 illustrates an embodiment of a bioreactor for producing nitric acid.

FIG. 10 illustrates an embodiment of a bioreactor for producing ammonium nitrate.

FIGS. 11A-11D illustrate different embodiments of a manure/composting housing or pile with different inlet feeds to produce different outlet feeds.

FIG. 12 illustrates an embodiment of an anaerobic digester.

FIG. 13 illustrates an embodiment of a manure tumbler.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein. Chemical substances are referred to herein by general names (e.g., ammonia) as well as chemical formula (e.g., NH3).

Generally, the present technology relates to microbially produced acids (e.g., microbial acids), mineral salts made therefrom (e.g., sulfur-containing mineral salt and/or nitrogen-containing mineral salts), and methods related to the selection of the microbes for producing the microbially produced acids and mineral salts. Microbial processing is any intake of a substance (e.g., sulfur containing substance or nitrogen containing substance) and processing that substance into a different substance, which can include any chemical or physical change to the substance as a result of the microbe interacting with the substance. As non-limiting examples, the microbes may produce sulfuric acid from sulfur containing substances or nitric acid from nitrogen containing substances. The sulfuric acid and nitric acid can be used in making mineral salts or for other uses. Non-limiting examples of mineral salts are sulfate, nitrates, and phosphates. Some examples of mineral salts can include sulfur-containing minerals and nitrogen-containing minerals, and minerals that contain both sulfur and nitrogen. The mineral salts can be used for various purposes, such as any use of the mineral salts that can be prepared with the bioreactors and methodologies in accordance with the disclosure provided herein. The mineral salts can be used for fertilizers, food processing, explosives, industrial applications, pharmaceuticals, or other use. The methods of producing the microbial acids may be considered to be organic methods that produce organic microbial acids, and thereby the mineral salts may be considered to be organic mineral salts. Thus, the processes described herein can provide products that can be certified as organic products.

By being microbially produced acids and corresponding mineral salts, such acids and mineral salts are biologically produced in the bioreactor under artificial conditions. Accordingly, any substance described herein to be produced by a microbe (e.g., microbially produced nitrogen-containing mineral) in a bioreactor with the reagent sources described herein may be referred to as being biologically produced because a natural biological process is used for making such a substance even through an artificial system is used for producing the substances. Thus, microbially produced acids may be referred to as biologically produced acids, and the corresponding mineral salts may also be biological mineral salts. However, it should be understood that the protocols are not natural protocols and use systems and reagents not natural to the production of the substances.

The processes described herein can use natural resources and natural reagents in natural processes in an artificial system for producing the microbial acids and mineral salts described herein. However, the natural resources and reagents may be provided in an artificial way in order to achieve the acids and minerals described herein. Accordingly, the processes can be devoid of using synthetic processes (e.g., synthetic chemical reactions), and thereby are non-synthetic in the sense of laboratory synthesis. In part, this is because the microbial acids are produced with microbes from natural and organic materials, such as animal waste or organic sulfurous acid or combinations thereof, or the like, and such microbial acids are considered to be non-synthetic. In another part, the microbial acids are combined with natural and organic materials (e.g., phosphate rock or animal waste) in order to produce the mineral salts in the artificial system. In another part, the processes described herein mimic and use natural processes for preparing the products in the artificial system. In another part, micro-nutrients may also be included in the processes so that the micro-nutrients may be included in the resulting mineral salts.

A sulfur containing substance is any molecule or element that includes sulfur in any fluid form (e.g., liquid or gas) or as a micron or nano-size particle, as sulfur or any compound including sulfur in it, that has the potential to be processed by microbes to result in a different molecule or element that includes sulfur. Examples of sulfur gases include H2S and SO2, and thereby any sulfur containing gas can include H2S and/or SO2. The methods can include using a sulfur containing substance in the selection of the microbes from microbes cultivated (e.g., obtained) from natural geological formations (e.g., geothermal areas) or unnatural conditions in an contaminated environment (e.g., mine waste contamination) where some portion of the different types of microbes process a sulfurous substance—different types of microbes may process different types of sulfur containing substances (e.g., different microbes being responsible for different steps in the processing into sulfuric acid). The methods can include using organically produced sulfurous acid in the production of sulfuric acid (e.g., MPSA). The methods can include using MPSA to form mineral salts, where the MPSA can be formed and then contacted with minerals or the MPSA can be formed in the presence of minerals. In one example method, the MPSA can be formed in the presence of ammonia to form an ammonium sulfate salt (e.g., (NH4)2SO4), such salt being similar to or the same as mascagnite. The MPSA can be used with any mineral or compound that reacts with an acid, such as sulfuric acid, to produce a mineral salt. Additional other methods of making or using MPSA or related methods are described herein.

The processes can include the use of microbes for preparing microbial acids and mineral salts. The microbes may be any type of microbe that is known or later found or developed. The microbes can be natural microbes or genetically modified microbes. The microbes can be of any type, such as bacteria, fungi, nematodes, algae, or any other microbial organism.

In one embodiment, the present technology relates to microbially produced sulfuric acid (MPSA; sulfuric acid produced as a result of microbial processing of sulfurous substances), mineral salts made therefrom, and methods related to the selection of the microbes. Microbial processing is any intake of a substance (e.g., sulfur containing substance) and processing that substance into a different substance, which can include any chemical or physical change to the substance as a result of the microbe interacting with the substance. A sulfur containing substance is any molecule or element that includes sulfur in any fluid form (e.g., liquid or gas) or as a micron or nano-size particle, as sulfur or any compound including sulfur in it, that has the potential to be processed by microbes to result in a different molecule or element that includes sulfur. The methods can include using a sulfur containing substance in the selection of the microbes from microbes cultivated (e.g., obtained) from natural geological formations (e.g., geothermal areas) or unnatural conditions in an contaminated environment (e.g., mine waste contamination) where some portion of the different types of microbes process a sulfurous substance—different types of microbes may process different types of sulfur containing substances (e.g., different microbes being responsible for different steps in the processing into sulfuric acid). The methods can include using organically produced sulfurous acid in the production of sulfuric acid (e.g., MPSA). The methods can include using MPSA to form mineral salts, where the MPSA can be formed and then contacted with minerals or the MPSA can be formed in the presence of minerals. In one example method, the MPSA can be formed in the presence of ammonia to form an ammonium sulfate salt (e.g., (NH4)2SO4), such salt being similar to or the same as mascagnite. The MPSA can be used with any mineral or compound that reacts with an acid, such as sulfuric acid, to produce a mineral salt. In some aspects, while using MPSA, the resulting mineral may or may not include sulfur, where the acid may facilitate production of the mineral without the sulfur being included in the mineral product. Additional other methods of making or using MPSA or related methods are described herein.

The use of MPSA can result in many different mineral salts depending on the composition of the other substances. The methods can include any methodology where MPSA is used to produce sulfate and phosphate mineral salts as well as nitrogen containing mineral salts. The MPSA can be used to manufacture any mineral salts that include phosphates, nitrates, and sulfates, such as for example and without limitation ammonium sulfate, potassium sulfate, potassium magnesium sulfate, potassium phosphate, and gypsum (e.g., hydrated calcium sulfate). The MPSA combined with other components of minerals can result in a mineral salt or combinations of mineral salts. These mineral salts can be used for supplying sulfur as a component of multi-nutrient salt in a sulfate form that is available for plant uptake. The mineral salts can include sulfur, nitrogen, phosphorus, potassium, and other minerals. Some examples of mineral salts that can be produced with the MPSA can include ammonium, phosphorus, or potassium salts; nitrogen-sulfur materials such as ammonium sulfate, ammonium nitrate-sulfate, ammonium phosphate-sulfate, and ammonium phosphate-nitrate; and potassium-sulfur salts such as potassium sulfate and potassium magnesium-sulfate. Sulfur is also a carrier for some of the micronutrients such as ZnSO4 and FeSO4, or others, which shows the MPSA can be used to produce such micronutrient salts as well. The micronutrients may also be contained within a produced mineral salt.

The use of MPSA can be to produce other microbial acids and salts thereof, depending on the other minerals or organic materials that are combined with the MPSA. For example, the methods can produce mineral salts such as or related to mascagnite, arcanite, anhydrite/gypsum, epsomite, or the like (e.g., Arcanite (K2SO4), Anhydrite/Gypsum (CaSO4), and Epsomite (Epsom salts) (MgSO4)). Now, any process for producing sulfuric acid can be replaced by using the MPSA, and any process using sulfuric acid can now use MPSA.

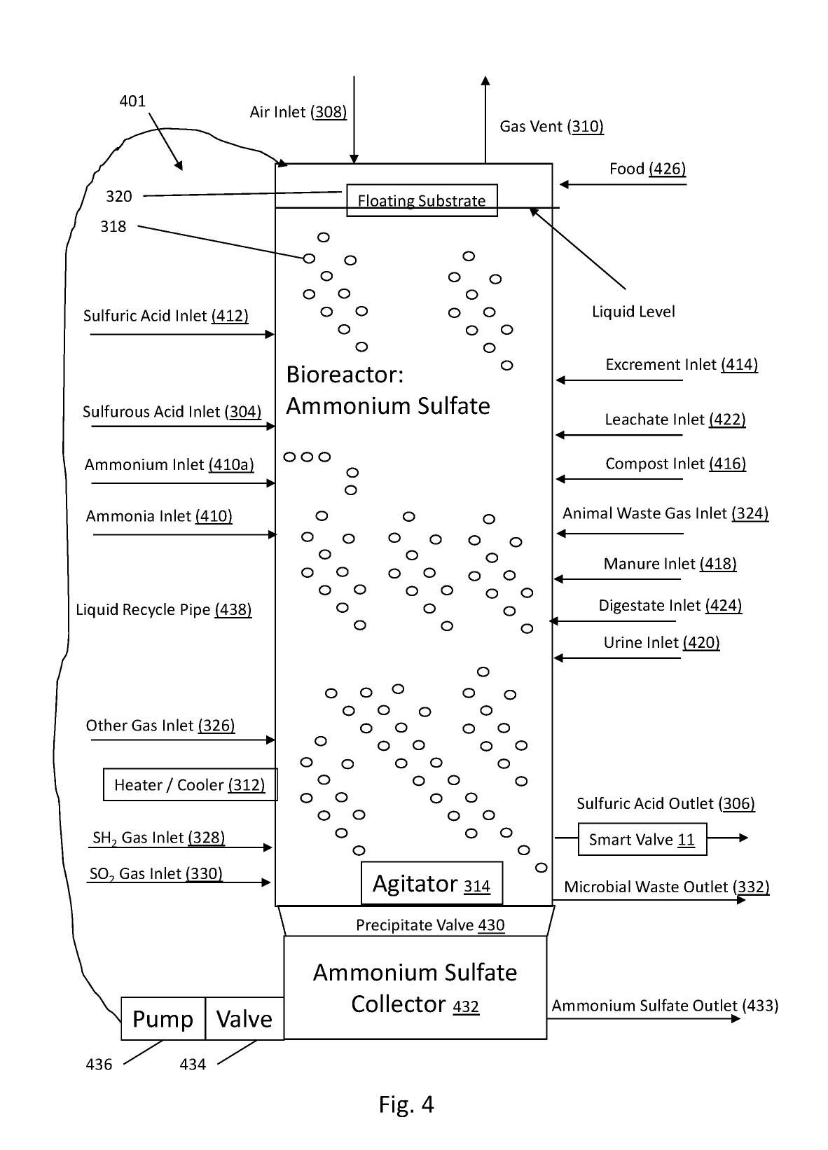

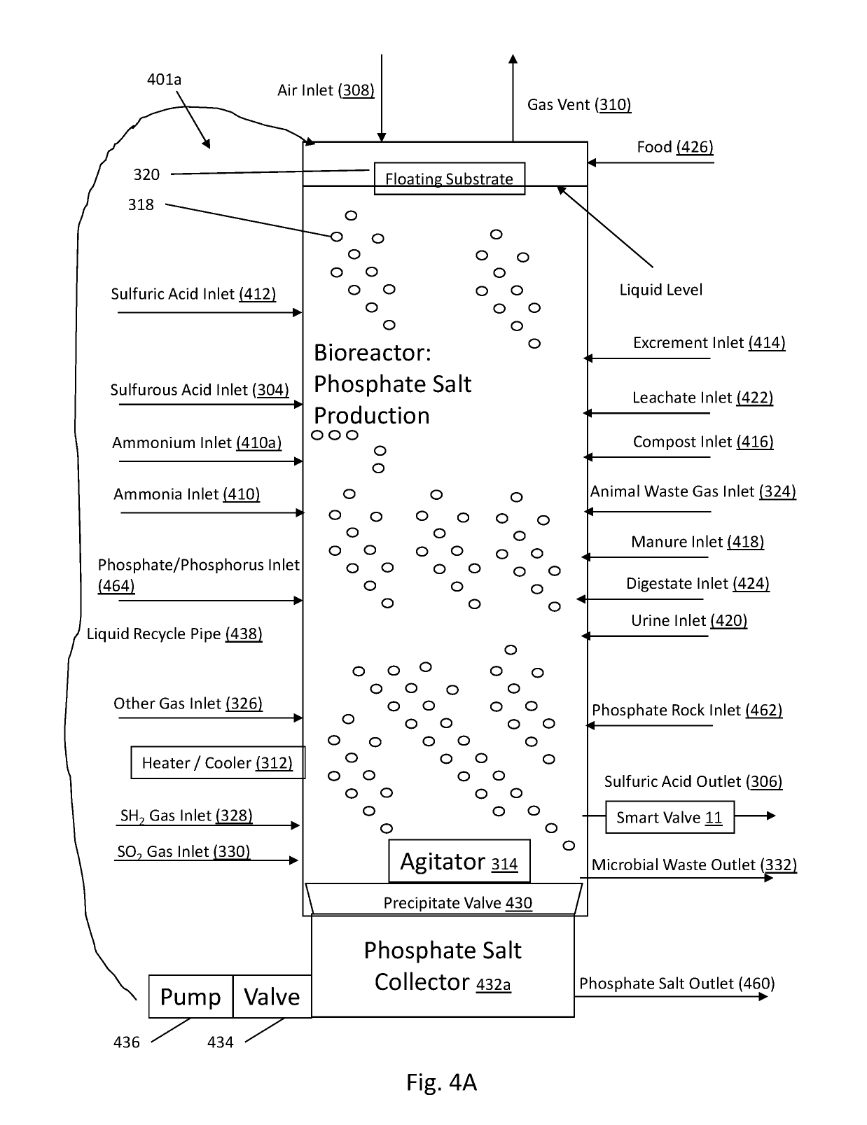

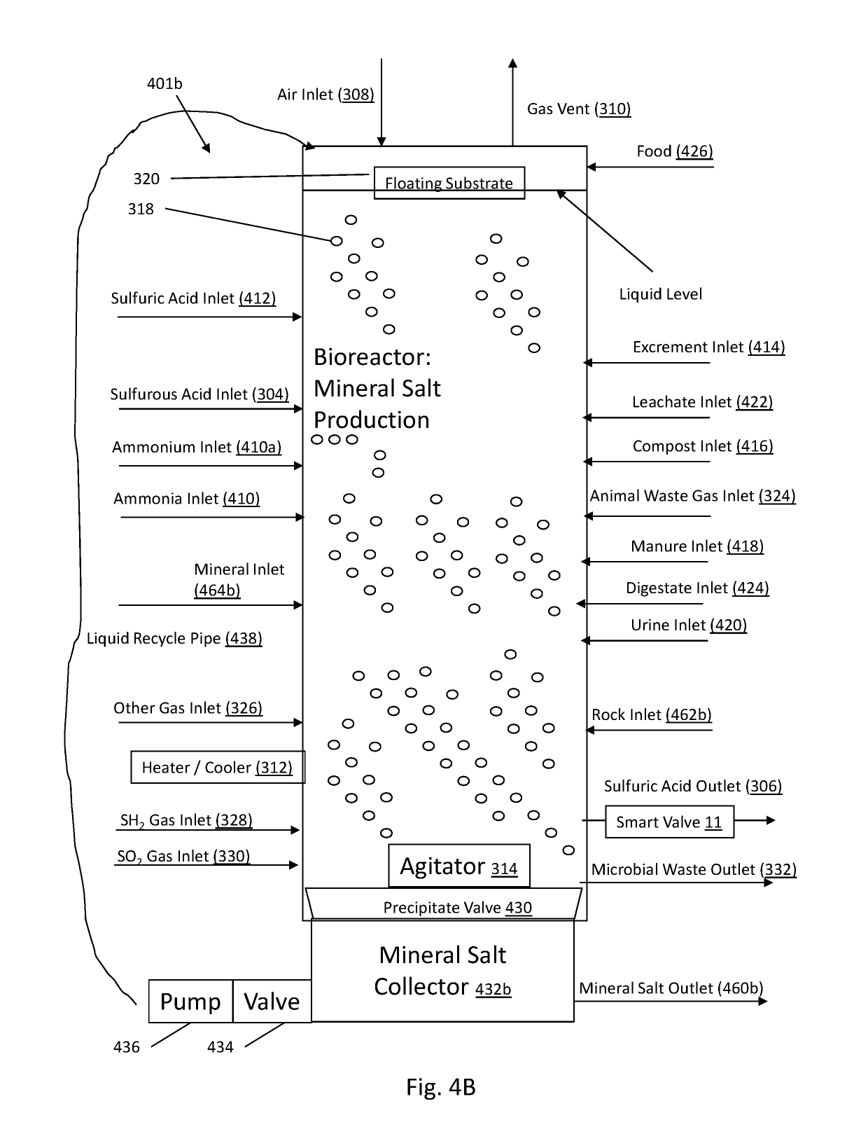

In one embodiment, the methods described herein can include microbial production of phosphoric acid via contacting phosphate rock with MPSA. That is, any method described herein can be supplanted by combining phosphate rock with MPSA or in the presence of the bioreactor forming MPSA to produce phosphoric acid or salt therefrom. Thus, for example bioreactors in FIGS. 4-4B can be modified to produce phosphoric acid or mineral salts thereof by introducing phosphate rock therein, such as FIG. 4A.

In one embodiment, the processes can be performed by capturing and using streams of NH3 and/or CO2 or any other gas emitted from a compost pile or barn or NH3 scrubber. Such captured streams can be used by mixing into HSO3 and sulfur-oxidizing microbes to produce ammonium salts or other nitrogen containing mineral salts.

In one embodiment, the processes can be performed by capturing and using streams of NH3 and/or CO2 or any other gas emitted from a compost pile or barn or NH3 scrubber and mix the captured streams with water and nitrifying microbes (e.g., nitrosomonas or any other) in order to produce nitrites (e.g., NO2). The nitrite can then be used to make HNO3 via any protonation. This process can form microbially produced nitric acid (e.g., MPNA). This organic MPNA can be used as the basis for even more concentrated forms of nitrogen containing mineral salts. Such a method can be used with any ammonia and/or carbon dioxide source, preferably organic sources.

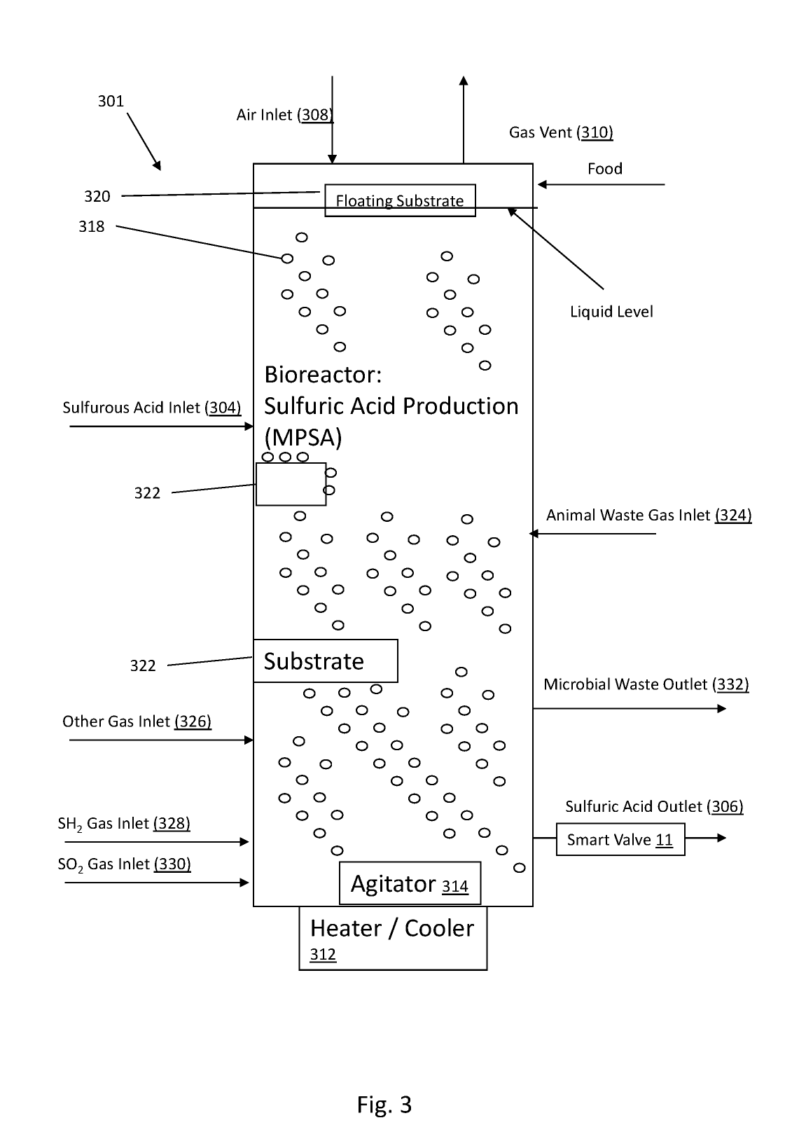

Accordingly, the methods described herein using sulfur-processing microbes to produce sulfuric acid (MPSA) may be modified by using nitrogen-processing microbes to produce nitrites that then are hydrogenated to produce nitric acid (e.g., MPNA), or other nitrogen containing compounds that can result in nitrogen containing minerals. The nitric acid can be a product, or it can be used in the formation of mineral salts. The changes in the bioreactors, reagents or input streams, or microbe populations in the bioreactors described herein (e.g., FIGS. 3, and 4-4A) can be made to use the nitrogen-processing microbes in addition to the sulfuric acid-producing microbes or to use the nitrogen-processing microbes instead of the sulfuric acid-producing microbes.

Also, any discussion of making or using sulfuric acid can be with the use of microbes, and the sulfuric acid can be MPSA. The use of mascagnite herein may generally refer to naturally occurring ammonium sulfate mineral, and vice versa.

In one embodiment, the technology describes using ammonia; however, such technology may be modified by using ammonia and ammonium or using ammonium without ammonia, or by using other nitrogen containing substances. For example, ammonium sulfate may be made with ammonium. The ammonium may also be obtained from the same sources as ammonia, and/or such ammonia may be hydrogenated to ammonia naturally, organically, or synthetically. Natural and organic production of mineral salts is desirable.

In one embodiment, the processes described herein can use select microbes in the production of sulfuric acid from a sulfur source. The technology also relates to the use of the produced sulfuric acid and an ammonia source for the production of ammonium sulfate, which can be exemplified by mascagnite. The examples herein can be modified by introducing other reagents to produce other sulfur containing minerals, nitrogen containing minerals, or minerals that contain nitrogen and sulfur, whether or not as ammoniums or sulfates or in other forms.

Mascagnite is a rare naturally occurring ammonium sulfate mineral (NH4)2SO4. Mascagnite crystallizes in the orthorhombic system typically forming as stalactitic masses exhibiting good cleavage. Mascagnite is soft (e.g., not higher than 2.5 on the Mohs scale) and water-soluble. Optical properties are variable; the purest form is transparent and colorless, but opaque gray or yellow deposits are also known. Mascagnite occurs in fumaroles, as at Mount Vesuvius and associated with coal seam fires. Now, mascagnite can be made by a microbial process that utilizes natural ammonia sources and sulfuric acid produced from microbes.

In one embodiment, microbes can be obtained by cultivation from a geothermal formation, such as a fumarole, hot pot, warm spring, geyser, hot vent, volcanic formation or any other geological formation that has a temperature that is warmer than the natural temperature or ambient temperature of its surroundings. Some examples of locations to obtain the microbes can include naturally occurring sulfuric volcanic crater lakes, such as at Kawha Lj en, Keli Mutu, Pinatubo, Chichon and any others.

In one embodiment, the microbes may be obtained from “snottites.” Snottite is a microbial mat of single-celled extremophilic bacteria that hang from the walls and ceilings of caves and are similar to small stalactites, but have the consistency of snot. In the Frasassi Caves in Italy, over 70% of cells in a snottite have been identified as Acidithiobacillus thiooxidans, with smaller populations including an archaeon in the uncultivated ‘G-plasma’ Glade of Thermoplasmatales (>15%) and a bacterium in the Acidimicrobiaceae family (>5%). These bacteria derive their energy from chemosynthesis of volcanic sulfur compounds including H2S and warm-water solution dripping down from above, producing sulfuric acid. Because of this, their waste products are highly acidic (approaching pH=0). Accordingly, such microbes from snottites may be used in the bioreactors described herein for the production of sulfuric acid or mineral salt, or any other use.

Additionally, the microbes may include or be supplemented with microbes that are found along with or on gypsum barite, naturally occurring sulfur, and many other natural sub stances/surfaces.

In one embodiment, microbes can be obtained by cultivation from a natural disaster area or an area of high contamination such as an old or abandoned mining site, which may or may not have elemental sulfur or sulfur dioxide. In one aspect, a superfund site, such as any mining site that has toxic water contaminated by various substances may be a source of the microbes. In these areas, such sites of cultivation can have acidic water that may be acidic from higher than normal levels of sulfuric acid. It is thought, without being bound thereto, that such areas can have acidic water from sulfuric acid produced by the microbes. The microbes can produce such sulfuric acid from a sulfur source, such as sulfur dioxide, H2S, or sulfides contained in the mined or exposed rock.

The cultivated microbes can be processed and selected for production of MPSA. That is, the microbes can be cultured and the culture media analyzed for sulfuric acid content and change in amount of sulfuric acid content over time as the microbes grow. The microbe cultures that increase sulfuric acid content can be selected for further processing and culturing. The microbe cultures that do not increase sulfuric acid content can be discarded. The assessment of sulfuric acid can be performed by any method, which can include taking a sample of the culture media and/or microbes and determining the acidity and sulfur content. The cultivated microbes can be cultured so that the microbe grow and propagate as is common for cultured microbes. The microbes can consume the sulfur, process the sulfur, oxidize or reduce the sulfur. The selection of the microbes can be performed by providing an environment (e.g., having sulfur) that is conducive for certain microbes to be cultured (e.g., microbes that process sulfur) and not conducive to other types of microbes, and thereby the microbes that grow can be selective.

The selected sulfuric acid producing microbes can be a collection of many microbes or a specific type. Often, the microbes will be aerobic. In other instances, the microbes can be anaerobic. Such anaerobic microbes can be bacteria, such as purple sulfur bacteria, which are a group of proteobacteria that are anaerobic or microaerophilic, and often found in hot springs or stagnant water, and may use hydrogen sulfide that is oxidized into elemental sulfur that is oxidized into sulfuric acid. The microbes may be bacteria that are involved in a sulfur cycle.

The environmental sulfur cycle involves many physical, chemical and biological agents, and is well known in the art. The relationships between sulfur, S, hydrogen sulfide, H2S, sulfur dioxide, SO2, and the sulfate ion, SO4 are well known, and utilized in the processes described herein. In mineral form sulfur may be present as sulfides (e.g. pyrite, FeS2, chalcopyrite, FeS.CuS, pyrrhotite, FeS) and/or any sulfates (e.g. gypsum,

CaSO4.2H2O, barite, BaSO4). Sulfur in minerals may move through the cycle as a result of the oxidation of sulfides to sulfate and/or the dissolution of sulfates. For example, oxidation of pyrite to sulfuric acid may be immediately followed, in situ, by acid neutralization by calcium carbonate (calcite) to form calcium sulfate (gypsum). The reaction of hydrogen sulfide with dissolved metal ions may precipitate metallic sulfides which are chemically indistinguishable from naturally occurring sulfide minerals. At some mines, sulfur is added to the cycle as sulfur dioxide in processes such as the Inco/SO2 process for cyanide destruction in the treatment of tailings. This added sulfur is oxidized to sulfate ion, most of which remains free, but some of which combines with lime, CaO, in the tailings to form gypsum.

Micro-organisms (e.g., bacteria, fungi, etc.) are often integrally involved in the chemical alteration of minerals. Minerals, or intermediate products of their decomposition, may be directly or indirectly necessary to their metabolism. The dissolution of sulfide minerals under acidic conditions like acid rock drainage (ARD), the precipitation of minerals under anaerobic conditions, the adsorption of metals by bacteria or algae, and the formation and destruction of organometallic complexes are all examples of indirect micro-organism participation. Where minerals are available as soluble trace elements, serve as specific oxidizing substrates, or are electron donors/acceptors in oxidation-reduction reactions, they may be directly involved in cell metabolic activity. There are three categories of oxidation-reduction reactions for minerals with micro-organisms: Oxidation by autotrophic (cell carbon from carbon dioxide) or mixotrophic (cell carbon from carbon dioxide or organic matter) organisms, where energy derived from the oxidation reaction is utilized in cell synthesis; Electron acceptance by minerals (reduction) for heterotrophic (cell carbon from organic matter) and mixotrophic bacteria, where chemical energy is used to create new cell material from an organic substrate; and Electron donation by minerals (oxidation) for bacterial or algal photosynthesis (reaction is fuelled by photon energy).

Oxidation of sulfur or sulfides for energy production can be from the bacterial genus Thiobacillus, the genus Thiomicrospira, and the genus Sulfolobus, or any others. These bacteria all produce sulfuric acid (i.e. hydrogen ions, H+, and sulfate ions, SO4-) as a metabolic product. These types of bacteria can accelerate the generation of ARD from sulfide bearing rocks under suitable conditions. The sulfide oxidation catalyzed by bacteria may have reaction rates six orders of magnitude (i.e. 1,000,000 times) greater than the same reactions in the absence of bacteria.

The direct reduction of sulfate ions to hydrogen sulfide is effected in nature by specialized, strictly anaerobic bacteria of the genera Desulfovibrio and\or Desulfotomaculum. These sulfate reducing bacteria (SRB) are heterotrophic (cell carbon from organic compounds) organisms that utilize sulfate, thiosulfate, S2O3-, sulfite, SO3-, or any other reducible sulfur-containing ions as terminal electron acceptors in their respiratory metabolism. In the process these sulfur-containing ions are reduced to hydrogen sulfide. The bacteria can use an organic substrate, which is usually a short chain acid such as lactic or pyruvic acid. In nature, such substrates are generated by the fermentation activities of other anaerobic bacteria on more complex organic substrates. Thus, in natural systems, the specific requirement for a short chain acid by the SRB is met by the availability of a complex organic source and a mixed bacterial system. This can be simulated in the processes described herein, such as by using animal excrement (e.g., solid and/or liquid) or compost.

The processing can be also done with: Ferric iron for sulfide oxidation; Thiobacillus ferrooxidans (T. ferrooxidans) and oxygen for ferrous to ferric oxidation; and at a pH compatible with T. ferrooxidans habitat requirements, typically pH 1.5-3.5.

Any microbe that can consume or process sulfur can be used in the process of producing sulfuric acid described herein. The microbes can be sulfur-reducing bacteria, which can obtain energy by processing organic compounds containing hydrogen (H2) while reducing sulfate to hydrogen sulfide. Many are anaerobes; however, there are sulfate-reducing bacteria that are tolerant of oxygen. The microbes can reduce other oxidized inorganic sulfur compounds, such as sulfite, thiosulfate, or elemental sulfur (e.g., which is reduced to hydrogen sulfide). In addition, there are sulfate-reducing bacteria that can reduce fumarate, nitrate and nitrite, iron (Fe(III)) and some other metals, dimethyl sulfoxide and even oxygen, wherein such bacteria can be used as described herein.

Additionally, the microbes can be Deltaproteobacteria such as the sulfate-reducing: Desulfobacterales, Desulfovibrionales and Syntrophobacterales. The sulfate-reducing bacteria can also include the Firmicutes, such as the genera Desulfotomaculum, Desulfosporomusa, and Desulfosporosinus. The microbes can be in the Nitrospirae division, such as sulfate-reducing Thermodesulfovibrio species. The microbes can include thermophile sulfate-reducing bacteria, such as the Thermodesulfobacteria and Thermodesulfobium. There are also Archaea that may be capable of sulfate reduction: Archaeoglobus, Thermocladium and Caldivirga, which may be found in hydrothermal vents, oil deposits, and hot springs.

In view of the foregoing, the microbes can be any microbe that processes sulfur in any way. Such microbes may process the sulfur from one state to another state. The sulfur in any state may be reactive and react with other substances in the production of sulfuric acid. Thus, any sulfur-processing microbe (e.g., SP microbe) can be used in the technology described herein. The microbes can be aerobic or anaerobic, known or yet to be identified. Thus, any microbe that facilitates either directly or indirectly the production of sulfuric acid can be used. In any event, selection of the microbes that facilitate production of sulfuric acid can be performed.

In one embodiment, the bacteria can be selected to produce ammonium sulfate from an ammonia source and a sulfur source. While it is thought that the bacteria produce sulfuric acid to combine with ammonia to produce ammonium sulfate, the production of ammonium sulfate may be from other processes performed by the selected bacteria. This production of ammonium sulfate can be in the presence of animal waste or compost that includes ammonia along with sulfur dioxide or sulfurous acid.

In one embodiment, the microbes are selected for sulfuric acid production by culturing the microbes in the presence of aqueous sulfur dioxide and/or sulfurous acid. A series of culture media can be prepared with increasing amounts of aqueous sulfur dioxide and/or sulfurous acid, and a colony of microbes can be cultured therewith. The colonies can be studied to see which ones have the best growth rates for reproduction and/or studied to see which ones cause the pH of the media to become more acidic from sulfuric acid production. The colonies that decrease the pH can be selected and propagated. Also, the selected microbes can be stored, and then used for production of sulfuric acid from aqueous sulfur dioxide and/or sulfurous acid in an organic system.

In one, embodiment, the microbes are selected for ammonium sulfate production by culturing the microbes in the presence of ammonia with aqueous sulfur dioxide and/or sulfurous acid. A series of culture media can be prepared with increasing amounts of ammonia, aqueous sulfur dioxide and/or sulfurous acid and combinations of ammonia and aqueous sulfur dioxide and/or sulfurous acid, and a colony of microbes can be cultured therewith. Also, media having any ratio from 1:10000 to 10000:1 ammonia to aqueous sulfur dioxide and/or sulfurous acid can be used for the selection. The colonies can be studied to see which ones have the best growth rates for reproduction and/or studied to see which ones cause the formation of ammonium sulfate. The colonies that produce the most ammonium sulfate can be selected and propagated. Also, the selected microbes can be stored, and then used for production of ammonium sulfate from ammonia with aqueous sulfur dioxide and/or sulfurous acid in an organic system.

In one embodiment, bacteria from the bacterial genus Thiobacillus, the genus Thiomicrospira, and/or the genus Sulfolobus can be provided for use in the processes described herein for the production of sulfuric acid and/or ammonium sulfate.

The microbes can be mesophilic, thermophilic, and acidophilic, whether mildly, moderately, or extremely. This can include microbes that can be cultured and/or thrive in cooler, intermediate, or hotter temperatures, as well as various pH ranges. These microbes can be cultivated or collected from any geothermal area, such as Yellowstone or Mammoth hot springs, or the like as well as any acid mine drainage or any natural or unnatural acid drainage into the environment. The collection can be via any standard sample collection, such as scooping an environmental sample, and then culturing the environmental sample under conditions suitable for sulfuric acid producing microbes to thrive.

In one embodiment, the microbes can be purchased and used as described herein.

In addition to the methods described herein or as a supplement thereto, the methods of making sulfuric acid can also use pyrite, solid sulfur, or any other forms of solid sulfur and apply the microbes to generate the H2SO4. However, in some embodiments use of solid sulfur is specifically excluded. Such excluded solid sulfur includes solid forms of sulfur, whether it is prilled, powder, flaked, ground, crushed ore, mined/crushed sulfide rocks and minerals, or other solid over a certain size. Examples of solid sulfur such as ground sulfur that are specifically excluded from the technology described herein (e.g., in some embodiments) include exclusion of any sulfur particle that is larger than 37 microns or larger than 74 microns in size, as it is noted that ground sulfur is usually at its smallest from common mechanical grinding facilities to be 37-74 microns.

On the other hand, the sulfur can be any pulverized, emulsions containing fine sulfur particles, and micron to nano sizes of sulfur. The size of any sulfur particle must be exceptionally small so as to not be considered a solid. The size of the sulfur should be small because the rate of conversion to sulfurous acid or sulfuric acid is inversely proportional to particle size of sulfur. The smaller the size the larger surface area that is provided to the microbes for processing, and thereby smaller sizes are more favorable. Thus, the sulfur particles used in the present technology should be less than 37 microns, or less than 25 microns, or less than 10 microns, or less than 1 micron, or less than 500 nm, or less than 250 nm, or less than 100 nm, or less than 50 nm, or less than 25 nm, or less than 10 nm, or less than 5 nm, or less than 1 nm, or less than 0.5 nm. Often, the sulfur will be a gas that is around 4 angstroms or 0.4 nanometers. In one aspect, the sulfur used in the processes can be in a liquid or gas format, such as aqueous sulfur. When aqueous, the sulfur particle size may be larger (e.g., larger than 37 microns), but it is preferred to get increased MPSA to use the smaller sizes recited in this paragraph. Examples of aqueous sulfur can include sulfur suspensions or emulsions with small sulfur particles as well as sulfurous acid or aqueous sulfur dioxide, or dihydrogen sulfide. Examples of gaseous sulfur can include sulfur dioxide or dihydrogen sulfide.

In one embodiment, the methods described herein can also include a step of determining the size of the sulfur particles used to in the bioreactors to make sulfuric acid or other sulfur-containing product. Such a method step can be a bright line test for ensuring small sulfur particles are obtained and used, such as produced by the burning of sulfur/sulfides, or its ores or by other methods for reducing the size of sulfur particles below normal sizes.

In addition to the microbes described herein or as a supplement thereto, the microbes may be from any source. The microbes can be selected for those that produce sulfuric acid. The microbes can be cultivated from any location. The microbes can be cultivated from any mineral or rock deposit that includes sulfur in the mineral or rock. In some examples, the microbes can be obtained from natural gypsum, barite, and any other substances/surfaces and utilized for producing MPSA as described herein.

In one embodiment, the production of sulfuric acid from microbes can be performed in a bioreactor. The bioreactor can have an input of aqueous sulfur dioxide and/or sulfurous, which can be from combustion of sulfur into sulfur dioxide that is combined with water to produce sulfurous acid. Any system and/or method for producing aqueous sulfur dioxide and/or sulfurous can be used to supply the same to the bioreactor containing the microbes, such systems or methods being described in U.S. Pat. No. 8,951,479; U.S. Pat. No. 7,767,162; U.S. Pat. No. 7,182,919; U.S. Pat. No. 6,689,326; U.S. Pat. No. 6,506,347; U.S. Pat. No. 6,500,391; U.S. Pat. No. 6,248,299; U.S. Pat. No. 6,080,368; U.S. RE-42,239; and U.S. 20003/0211018 or U.S. provisional 62/160,980, each of which is incorporated herein by specific reference. The sulfurous acid can include H2SO3, and may be any combination of H2O, SO2, H+, HSO3, SO3, and/or H2SO3. In one aspect, trickle reactors with SO2 gas being pushed into the reactor may be considered as another form of H2SO3 or equivalent, since it will contact water trickling down and form H2SO3.

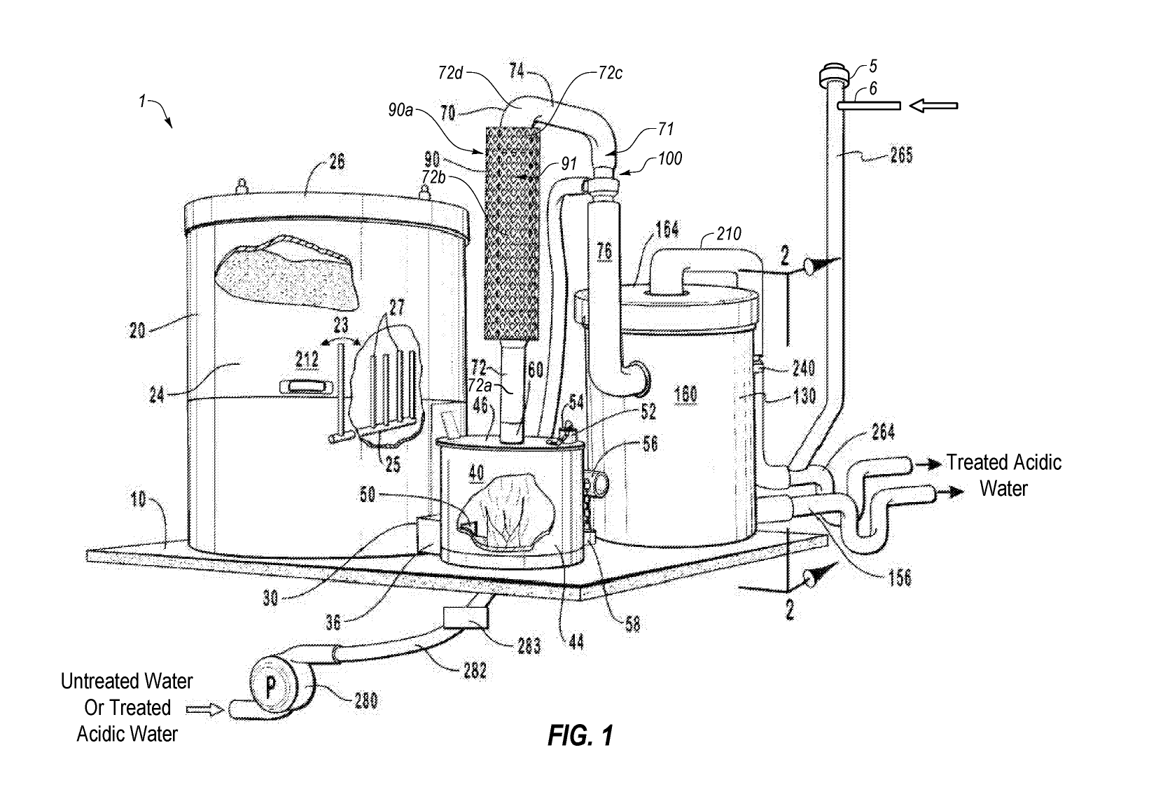

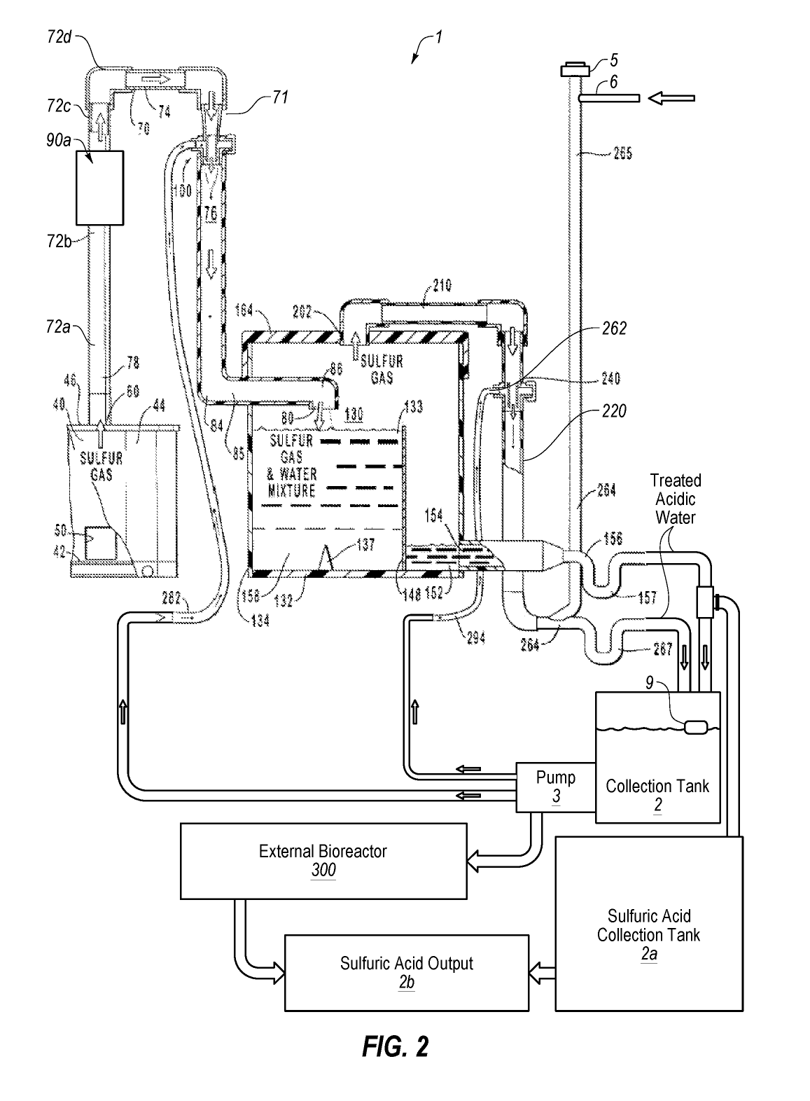

In one embodiment, the aqueous sulfur dioxide and/or sulfurous acid can be prepared by a system as illustrated in FIGS. 1 and 2. A system for preparing sulfurous acid (which can refer to aqueous sulfur dioxide and/or sulfurous acid) includes a burn chamber with a vertical exhaust pipe extending from the burn chamber, where the vertical pipe has an upper section configured to implement a secondary burn. The primary burn is in the burn chamber, and as burn exhaust travels up the vertical exhaust pipe, a secondary burn occurs. In one embodiment, the secondary burn occurs in a secondary burn region of the vertical exhaust pipe, where the vertical exhaust pipe is shaped like a candy cane with the lower column of the candy cane attached to the primary burn chamber, where the lower column extends vertically through a middle column section then to an upper column section before bending around and then pointing downward like a candy cane. The upper bend section of the candy cane is considered to be the traverse pipe and the portion that points downwardly again is the down pipe. The secondary burn region can occur in any section of the candy cane. However, the secondary burn region can advantageously be in the middle column section or upper column section. In some instances the secondary burn region can be in the traverse pipe. In any embodiment, the second burn region can be implemented in any sulfurous acid production system, such as shown in FIGS. 1 and 2. However, any system that produces aqueous sulfur dioxide and/or sulfurous acid can be used.

FIGS. 1 and 2 illustrate examples of a sulfurous acid generating system 1 which generates sulfur dioxide gas and keeps the gas substantially contained and in contact with water for extended periods of time, substantially eliminating any significant release of harmful sulfur dioxide gas from the system. As shown in FIG. 1, the system can include a sulfur hopper 20, a burn chamber 40, an exhaust gas pipeline or first conduit 70, a mixing tank 130, and an exhaust conduit 210. The principle elements can be mounted on a platform 10 to facilitate transport. FIG. 1 shows an embodiment of the system that can be modified as described herein. As such, the outlet of treated acidic water, such as from 156 and 264 can be emptied into a common tank, and then pumped with one or more pumps back to the venturi pump, such as venturi pump 100 and venturi pump 240 as well as any additional venturi, such as the third, fourth, fifth, or more venturi pump.

In one embodiment, weir 148 of the figures can be removed so that there is no spill over. The configuration without the weir 148 can operate as described herein with the gases going out exhaust vent 202 and the liquid going out aperture 154 to drainage pipe outlet 156.

The sulfur hopper 20 can include an enclosure 24 with a top surface 26. The top surface 26 defines a closeable aperture, not shown, and thereby top surface 26 can be a lid. Enclosure 24 may be of any geometric shape; cylindrical is shown, rectangular may also be employed. Surface 26 of enclosure 24 can include a closeable aperture of sufficient diameter and shape to allow sulfur to be loaded into the hopper 20. The enclosure 24 defines a hopper outlet 30. The hopper 20 is configured such that sulfur in the hopper 20 is directed toward the hopper outlet 30 by the pull of gravity. The hopper outlet 30 allows sulfur to pass through and out of the hopper 20.

An optional passageway conduit 36 communicates between the hopper outlet 30 and a burn chamber inlet 50 of the burn chamber 40. While hopper outlet 30 is shown to be ported into the bottom of the burn chamber 40, the burn chamber inlet 50 can be at the middle of the side, top of the side, or into the top of the burn chamber 40.

In an alternative embodiment, enclosure 24 may include a rocker arm 23 mounted external of enclosure 24. Rocker arm 23 is capable of being moved back and forth as shown by the arrow under the number 23. Connected to substantially vertical rocker arm 23, and extending internal to enclosure 24, is bar 25 having substantially upward extending fingers 27. Fingers 27 extend upward into the sulfur supply such that rocker arm 23 can be manipulated back and forth, thereby manipulating or raking fingers 27 back and forth to aviate incavitation of sulfur that may occur in hopper 20. This also allows for stirring or moving the sulfur supply so that it does not get lodged in the hopper 20. A mechanical system can be coupled to the rocker arm 23 to facilitate rocking of the sulfur in the hopper 20 The burn chamber 40 can include a floor member 42, a chamber sidewall 44 and a roof member 46. The floor member 42 defines a perimeter and the floor member 42 can be removably attached to the chamber sidewall 44. The roof member 46 is secured to the chamber sidewall 44, the chamber sidewall 44 supporting the roof member 46. The roof member 46 can also be a removable lid. An ignition inlet 52 defined by the roof member 46 has a removably attached ignition inlet cover 54. An air inlet 56 defined by the chamber sidewall 44 has a removably attached air inlet cover 58. The air inlet 56 is positioned substantially opposite to the burn chamber inlet 50 and may enter the chamber sidewall 44 tangentially or at the bottom, middle or top of the burn chamber 40. An exhaust opening 60 in the burn chamber 40 is defined by the roof member 46. The air can be ambient air. The air may also be condensed and pressurized.

Sulfur supplied to the burn chamber 40 through the burn chamber inlet 50 can be ignited through the ignition inlet 52. The air inlet 56 allows oxygen, necessary for the combustion process, to enter into the burn chamber 40, and thus permits regulation of the rate of combustion. The exhaust opening 60 allows the sulfur dioxide gas to pass up through the exhaust opening 60 and into the exhaust gas or first conduit pipeline 70.

The exhaust gas pipeline or first conduit 70 has two ends, the first end 78 (e.g., at the lower column section) communicating with the exhaust opening 60, the second end 71 terminating at a third conduit 76. The gas pipeline or first conduit 70 may comprise an ascending pipe 72 having the lower column section 72a, middle column section 72b, and upper column section 72c that turns at the bend 72d to a transverse pipe 74. The ascending pipe 72 may communicate with the transverse pipe 74 by means of a first 90 degree elbow joint at the bend 72d.

Disposed about and secured to the ascending pipe 72 is secondary burn member 90 to prevent cooling of ascending pipe 72 which is hot when in use. The secondary burn member 90 can be various entities that promote a secondary burn within the ascending pipe 72. For example, the secondary burn member 90 can be any entity that retains heat or increases heat, such as an active heating element, electrical heating element, insulator, or the like. In one aspect, the secondary burn member 90 is an insulator that retains heat in the ascending pipe 72. The secondary burn member 90 can be added to any of the ascending pipes or candy cane pipes described herein so as to form a secondary burn chamber 91.

In one embodiment, adding insulation 90a to the pipe 72 above the burn chamber can create a secondary burn chamber 91 in the ascending pipe 72 triggered by an increase in insulated pipe temperature. This secondary burn chamber 91 can be sufficiently separated from the burn chamber 40 so that the burn chamber temperature is not affected by secondary chamber heat. If the primary burn chamber temperature gets too high, there can be excessive boiling of the sulfur and not enough oxygen to create the desired sulfur dioxide. The length of the insulation 90a depends on the air flow into the burn chamber 40. The desired temperature of the secondary burn chamber 91 is about 450-1200 degrees F. (232-649 degrees C.); however, the temperature can be modulated as described in the incorporated references.

The insulation 90a can have various parameters so long as it causes formation of the secondary burn chamber 91 in the ascending pipe 72 that is covered by the insulation 90a. The insulation can be any standard hot pipe insulation that can withstand elevated temperatures.

In practice, it was found that the secondary burn chamber 91 is formed during the process, and presented as the upper column section 72c is glowing red. The desired temperature of the secondary burn chamber 91 is at or greater than 400 degrees F. (204 degrees C.), or about 450-1500 degrees F. (232-850 degrees C.), or 500 to 1200 degrees F. (260-649 degrees C.); or 600-1000 degrees F. (315-538 degrees C.); however, the temperature can be modulated as described herein. The sulfur dioxide gas can be in the secondary burn chamber 91 for approximately 0.3 seconds, 0.2-0.4 seconds, or 0.1-0.5 seconds.

In one aspect, the secondary burn chamber 91 can also be achieved by regulating the flow rate of exhaust gas from the burn chamber 40 through the ascending pipe 72, where a decrease in flow rate can improve the secondary burn. The flow rate of exhaust gas in the ascending pipe 72 can also be regulated by modulating the flow rate in the first venturi. The modulation of flow rate of the venturi will be described in more detail below.

The secondary burn chamber 91 can operate because not all of the sulfur supply burns in the burn chamber 40. As such, sulfur particles can be entrapped by the exhaust from the burn chamber 40 and travel up into the ascending pipe 72, which is not favorable. The excess sulfur particles in the exhaust can cause problems for the process and piping, which is described herein. As such, implementing the secondary burn chamber 91 can burn the sulfur in the exhaust to cause improved sulfur burning and improved generation of sulfur dioxide in the exhaust flow.

The flow of exhaust gas in the ascending pipe 72, and thereby in the secondary burn chamber 91, can be between 20 and 400 gallons of exhaust per minute. However, modulating the exhaust flow rate to between 30 to about 350 gallons per minute, or 40 to about 300 gallons per minute, or 50 to about 250 gallons per minute can improve the combustion to fuller combustion in the secondary burn chamber 91. Also, it should be recognized that the flow rate can be varied to any value of these ranges. Also, it should be recognized that larger systems may have flow rates higher than 400 gallons per minutes.

During the process, water is conducted through a second conduit 282 to a point at which the second conduit 282 couples with the first conduit 70 at a third conduit 76. The point can be a venturi pump 100. Third conduit 76 can include a co-directional flow means 100 (e.g., venturi pump) for bringing the sulfur dioxide gas in the first conduit 70 and substantially all the water in second conduit 282 into contained co-directional flow, whereby the sulfur dioxide gas and water are brought into contact with each other in the venturi pump 100.

The codirection flow means 100 allows water to be introduced into the third conduit 76 initially through a second conduit inlet. The water entering the codirectional means 100 passes through the eductor and, exits adjacent the sulfur dioxide gas outlet.

The water enters the third conduit 76 and comes into contact with the sulfur dioxide gas exiting by surrounding the sulfur dioxide gas where the sulfur dioxide gas and water are contained in contact with each other. The water and sulfur dioxide gas react to form an acid of sulfur-sulfurous acid. This first contact containment portion of conduit 76 does not obstruct the flow of the sulfur dioxide gas. It is believed that a substantial portion of the sulfur dioxide gas will react with the water in this first contact containment area.

After the acid and any host water (hereafter “water/acid”) and any remaining unreacted gas continue to flow through third conduit 76, the water/acid and unreacted sulfur dioxide gas are mixed and agitated to further facilitate reaction of the sulfur dioxide with the water/acid. Means for mixing and agitating the flow of water/acid and sulfur dioxide gas is accomplished in a number of ways. For example, mixing and agitating can be accomplished by changing the direction of the flow such as a bend 84 in the third conduit 76. Another example includes placing an object, such as a baffle or tornado, inside the third conduit 76 to alter the flow pattern in the third conduit 76. This could entail a flow altering wedge, flange, bump or any other member along the codirectional flow path in third conduit 76. By placing an object in the flow path, a straight or substantially straight conduit may be employed. The distinction of this invention over the prior art is mixing and agitating the flow of water/acid and sulfur dioxide involving substantially all of the water of the system with sulfur dioxide gas in an open codirectionally flowing system. Another way to increase contact of the sulfur dioxide gas and water/acid is to include multiple codirection flow means 100 in sequence, with the outlet of one codirection flow means 100 being the inlet of the next codirection flow means 100. Any number of codirection flow means 100 can be in sequence, as described herein. One embodiment of the present invention can treat between 20 and 400 gallons of water per minute coursing through third conduit 76 being held in contained contact with the sulfur dioxide gas.

After the water/acid and sulfur dioxide gas have passed through an agitation and mixing portion of third conduit 76, the water/acid and unreacted sulfur dioxide gas are again contained in contact with each other to further facilitate reaction between the components to create an acid of sulfur. This is accomplished by means for containing the water/acid and sulfur dioxide gas in contact with each other. One embodiment is shown as a portion 85 of third conduit 76. Portion 85 acts in much the same way as the earlier described contact containment portion.

In one embodiment, additional means for mixing and agitating the codirectional flow of water/acid and sulfur dioxide gas is employed. One embodiment is illustrated as portion 86 of third conduit 76 in which again the directional flow of the water/acid and sulfur dioxide gas is directionally altered. In this way, the water/acid and sulfur dioxide gas are forced to mix and agitate, further facilitating reaction of the sulfur dioxide gas to further produce or concentrate an acid of sulfur.

Third conduit 76 also incorporates means for discharging the water/acid and unreacted sulfur dioxide gas before the various third conduits 76. One embodiment is shown in FIG. 2 as discharge opening 80 defined by third conduit 76. Discharge opening 80 is preferably positioned approximately in the center of the pooling section, described below. In one embodiment, discharge 80 is configured so as to direct the discharge of water/acid and unreacted sulfur dioxide gas downward into a submersion pool 158 without creating a back pressure. In other words, discharge 80 is sufficiently close to the surface 133 of the fluid in the submersion pool to cause unreacted sulfur dioxide gas to be forced into the submersion pool, but not below the surface of the fluid in the submersion pool, thereby maintaining the open nature of the system and to avoid creating back pressure in the system.

The present invention also utilizes a mixing tank 130 having a bottom 132, a tank sidewall 134, and a lid 164. While referred to as a mixing tank 130, it may well be considered a separation tank because the gas is separated from the liquid, or a collection tank to collect the acidic water. Tank 130 may also comprise a fluid dispersion member 137 to disperse churning sulfurous acid and sulfur dioxide gas throughout tank 130. Dispersion member 137 may have a conical shape or any other shape which facilitates dispersion. An optional weir 148 may be attached on one side to the bottom member 132 and is attached on two sides to the tank sidewall 134. The weir 148 extends upwardly to a distance stopping below the discharge 80. The weir 148 divides the mixing tank 130 into a submersion pool 158 and an outlet section 152. The third conduit 76 penetrates the tank sidewall 134 at a point below the lid 164. An outlet aperture 154 is positioned in the tank sidewall 134 near the bottom member 132 in the discharge section. The outlet aperture 154 is connected to an outlet 156. Outlet 156 is adapted with a u-trap 157. U-trap 157 acts as a means to force levels of undissolved gas of sulfur dioxide gas back into chamber 130 to exit through lid 164 into vent conduit 210. The treated acidic water from outlet 156 can be provided to a collection tank and then pumped back to the venturi pump 100 and/or venturi pump 240 as well as any other additional venturi pumps.

The system can implement recycling from the discharge 264 and outlet 156 to a common collection tank. The common collection tank then provides the treated acidic water to a pump that pumps the treated acidic water to the venturi pump 100 and venturi pump 240 as well as an additional venturi pumps.

As sulfurous acid flows out of the third conduit 76, the weir 148 dams the acid coming into the mixing tank 130 creating a churning submersion pool 158 of sulfurous acid. Sulfur dioxide gas carried by but not yet reacted in the sulfurous acid is carried into submersion pool of acid 158 because of the proximity of the discharge 80 to the surface 133 of the submersion pool of acid 158. The carried gas is submerged in the churning submersion pool of acid 158. The suspended gas is momentarily churned in contact with acid in submersion pool of acid 158 to further concentrate the acid. As unreacted gas rises up through the pool, the unreacted gas is held in contact with water and further reacts to further form concentrate sulfurous acid. The combination of the discharge 80 and its close proximity to the surface 133 of submersion pool of acid 158 creates a means for facilitating and maintaining the submersion of unreacted sulfur dioxide gas discharged from the third conduit 76 into the submersion pool of sulfurous acid to substantially reduce the separation of unreacted sulfur dioxide gas from contact with the sulfurous acid to promote further reaction of the sulfur dioxide gas in the sulfurous acid in an open system without subjecting the sulfur dioxide gas discharged from the third conduit to back pressure or system pressure. That is, discharge 80 positioned below the level of the top of weir 148 is contemplated as inconsistent with an open system.

Any free floating sulfur dioxide gas in mixing tank 130 rises up to the lid 164. The lid 164 defines an exhaust vent 202. Exhaust vent 202 may be coupled with an exhaust vent conduit 210. The exhaust vent conduit 210 has a first end which couples with the exhaust vent 202 and a second end which terminates at a fourth conduit 220. The exhaust vent conduit 210 may consist of a length of pipe between vent 202 and the fourth conduit 220. The fourth conduit 220 comprises auxiliary means 240 (e.g., auxiliary venturi pump) for bringing sulfur dioxide gas in the exhaust vent conduit 210 and substantially all the water in a supplemental water conduit hose 294 into contained, codirectional whereby remaining sulfur dioxide gas and water are brought into contact with each other.

The auxiliary means 240 (e.g., venturi pump) has a body defining a gas entry, a gas outlet, a supplemental water conduit inlet, and water eductor. The auxiliary means 240 can be configured as the venturi pump 100.

Water enters the auxiliary means 240 through the supplemental water conduit 294 at supplemental water conduit inlet. The water courses through the water eductor as discussed earlier as to the codirectional means. The water eductor draws any free floating sulfur dioxide gas into the exhaust vent conduit 210. Water and sulfur dioxide gas are brought into contact with each other in fourth conduit 220 by surrounding the gas with water. The water and gas are contained in contact with each other as the gas and water flow down through fourth conduit 220 to react and form an acid of sulfur. This contact containment area does not obstruct the flow of the sulfur dioxide gas. Substantially all of the sulfur dioxide gas in exhaust vent conduit 210 reacts with the water in this contact containment area.

In fourth conduit 220, the water/acid and unreacted or undissolved sulfur dioxide gas also experience one or more agitation and mixing episodes. For example, as water reenters fourth conduit 220 at inlet 262, the flow of water/acid and sulfur dioxide gas is mixed and agitated. The water/acid and sulfur dioxide gas are again contained in contact with each other thereafter. Another similar mixing and agitating episode occurs when the directional flow of the water/acid and sulfur dioxide gas is altered near discharge 264. As a result, like the water/acid and sulfur dioxide gas in the third conduit 76, the water/acid and sulfur dioxide gas in fourth conduit 220 may be subject to one or more contact containment portions and on or move agitation and mixing portions. The fourth conduit may have a u-trap 267. U-trap 267 acts as means to cause bubbles of unabsorbed diatomic nitrogen gas to be held on the upstream side of u-trap 267. Discharge 264 is also configured with a vent stack 265. Remaining diatomic nitrogen gas in the system is permitted to escape the system through vent stack 265. Operation of the system reveals that little, if any, sulfur dioxide escapes the system. It is believed that gas that is escaping the system is harmless diatomic nitrogen. This configuration of a sulfur acid generator eliminates the structure, expense and use of a counter current absorption tower of the prior art. The treated acidic water from discharge 264 can be provided to a collection tank and then pumped back to the venturi pump 100 and/or venturi pump 240 as well as any other additional venturi pumps. As such, the treated acidic water from the discharge 256 can be combined with the treated acidic water from outlet 156, where both can be provided to a collection tank and then pumped back to the venturi pump 100 and/or venturi pump 240 as well as any other additional venturi pumps.

It will be appreciated that any pump capable of delivering sufficient water to the system may be utilized and the pump may be powered by any source sufficient to run the pump. A single pump with the appropriate valves may be used or several pumps may be used. It is also contemplated that no pump is necessary at all if an elevated water tank is employed to provide sufficient water flow to the system or if present water systems provide sufficient water pressure and flow.

In one embodiment, with reference to FIG. 1, the vent stack 265 can include a gas flow reducer 5. The gas flow reducer 5 can completely inhibit gas flow out the top of the chimney so as to function as a cap. However, gas pressure may build to cause problems, and thereby the gas flow reducer 5 can be configure to reduce the gas flow or to inhibit gas flow until reaching a certain pressure. The gas flow reducer 5 can be configured as a cap with small holes to reduce gas flow out of the vent stack 265. The gas flow reducer 5 can also be configured with a pressure release valve that releases gas when the pressure in the chimney reaches a predetermined. The vent stack 265 may also include a water inlet 6, which can supply untreated water or treated sulfurous acid water to the vent stack 265. This can be used to scrub the exhaust and increase efficiency of sulfurous acid production from the system 1.

FIG. 1 shows an embodiment of the system that shows recycling from the discharge 264 and drainage pipe outlet 156 to a common collection tank 2 for collecting sulfurous acid (e.g., sulfurous acid collection tank). The common collection tank 2 then provides the treated acidic water to a pump 3 that pumps the treated acidic water to the venturi pump 100 and venturi pump 240 as well as an additional venturi pumps. Also, while no outlet is shown, it is well within the skill in the art to have an outlet anywhere sulfurous acid is present, such as in tanks (e.g., collection tank 2) or from 166 and 264, or anywhere a pH sensor identifies the desired pH, and the system can include valves and outlets for such withdrawal. The collection tank 2 can, with or without the pump 3, provide sulfurous acid to the external bioreactor 300. Sulfuric acid can be prepared in the external bioreactor 300 as described herein, and sulfuric acid can be provided by a sulfuric acid output 2b.

Also, the outlet from drainage pipe outlet 156 can flow to a smart valve 11, which can open and allow sulfuric acid to flow into the sulfuric acid collection tank 2a. The sulfuric acid can also be provided at the sulfuric acid output 2b.

In one embodiment, the system shown in FIGS. 1 and 2 can be configured to produce sulfuric acid. While the system has been described as producing sulfurous acid, various modifications to the system can results in production of sulfuric acid.

A first system modification can include using the tank 130 as a bioreactor 160 to include the sulfuric acid producing microbes. That is, the microbes can be cultured in the tank 130 so that the microbes produce sulfuric acid from the aqueous sulfur dioxide and/or sulfurous acid. Experiments have shown that the cultivated microbes can be cultured in the presence of aqueous sulfur dioxide and/or sulfurous acid to produce sulfuric acid. As such, the microbes can be cultured in the tank 130 when it is operated as a bioreactor. The bioreactor may or may not include additional food for the microbes to consume for culturing. It has been found that no additional food for microbe consumption is needed for the microbes to produce the sulfuric acid. However, there may be instances when the microbes may utilize an additional food source to facilitate sulfuric acid production, which may be the sulfuric acid producing microbes or companion microbes (e.g., ones that do not actively produce sulfuric acid but that exist in a cooperative environment with the microbes that produce the sulfuric acid) consuming the food source. In any event, the microbial population can produce sulfuric acid in the tank 130 when functioning as a bioreactor. The tank 130 may be operated as is known for bioreactors, which may optionally include supplying air into the tank as well as carbon dioxide. When sulfuric acid is produced in the tank 130, it can be collected at outlet 156, and used as described herein or stored. Optionally, the sulfuric acid along with any aqueous sulfur dioxide and/or sulfurous acid may be recycled as described herein. A valve at 156 can be controlled so that when the outlet 156 has mainly aqueous sulfur dioxide and/or sulfurous acid, then there is recycling, but when the pH drops below a certain level associated with sulfuric acid, then the valve can cause the outlet 156 to stop the recycling and to provide the sulfuric acid for use or storage. Accordingly, the treated acidic water can be aqueous sulfur dioxide and/or sulfurous acid and/or sulfuric acid, and preferably include sufficient sulfuric acid when tank 130 is a bioreactor with sulfuric acid producing microbes.

As shown in FIG. 2, the outlet 156 may be fluidly coupled to a smart valve 11 that has outputs to the sulfurous acid collection tank 2 and the sulfuric acid collection tank 2a. The smart valve 11 can be a valve that selectively changes the flow between the sulfurous acid collection tank 2 and sulfuric acid collection tank 2a based on various parameters or instructions. In one instance, a controller that controls the system can provide instructions to change the output between these collection tanks. In another instance, the smart valve 11 can include a pH meter that once a certain pH threshold is met, then the valve redirects the output. For example, if below the pH threshold, the smart valve 11 directs the output to the sulfuric acid collection tank, but when above the pH threshold, the smart valve 11 directs the output to the sulfurous acid collection tank 2. Such a smart valve 11 allows for recycling the sulfurous acid to increase acidity (e.g., decrease pH) until there is sufficient sulfuric acid, and then the sulfuric acid can be withdrawn into the sulfuric acid collection tank 2a until there isn't sufficient sulfuric acid, and then the output can be directed back to the sulfurous acid collection tank for recycling.

FIG. 2 also shows that the sulfurous acid collection tank 2 can by a pump 3 or gravity feed supply the sulfurous acid into an external bioreactor 300. The external bioreactor 300 can include the microbes that produce sulfuric acid and may be operated substantially similarly to the bioreactor of tank 130. The external bioreactor 300 can then provide a sulfuric acid output that can be used or stored as described herein. The sulfuric acid collection tank 2a can also provide the sulfuric acid output, and both the external bioreactor and sulfuric acid collection tank 2a can produce separate sulfuric acid output or be combined to produce a common sulfuric acid output 2b. A smart valve may also be used to determine when the external bioreactor 300 can provide the sulfuric acid output, such as when the pH drops below a threshold as described herein. This allows either batch or continuous generation of sulfuric acid from microbe production.

Additionally, the systems of FIGS. 1 and 2 can be modified to produce other acids (e.g., nitrogen containing acids) when including nitrogen processing microbes. Also, the systems of FIGS. 1 and 2 can be modified to produce any of the mineral salts described herein by including the reagents or elements to be included in the minerals as well as microbes that can process the sulfur, nitrogen, or other elements so that the substances of the minerals is produced in a manner that allows for the minerals to be formed. The minerals that are formed can be collected from the tank 130 or any other tank. Also, the tank 130 may be modified into any of the bioreactors described herein in order to use tank 130 as a bioreactor that produces the acids and the mineral salts that include sulfur and/or nitrogen.

It should be noted that under FIGS. 1 and 2 that some of the sulfur may not be burned in the process, which can result in some elemental sulfur. As such, the collection tank 2 can include some sulfur along with the sulfur dioxide gas and sulfurous acid. Accordingly, some elemental sulfur may be collected, and may be introduced into any of the bioreactors described herein, such as when introducing the sulfuric acid or sulfurous acid.

FIG. 3 illustrates an example of a bioreactor 301 configured for sulfuric acid production from microbes. The bioreactor 301 can be the mixing tank 130 or external bioreactor or any other bioreactor described herein that includes sulfuric acid producing microbes. The bioreactor 301 can include a sulfurous acid inlet 304 and sulfuric acid outlet 306, which can be at any location with respect to the top and/or bottom, or as shown. The bioreactor 301 can include an air inlet 308 and a gas vent 310, which allows the bioreactor 301 to operate at ambient conditions. The bioreactor 301 can include a heater and/or cooler 312 for temperature control, or it can be operated at ambient temperatures. It can be heated to any temperature suitable or favorable for the microbes to produce sulfuric acid, which temperature can be modulated in order to determine the optimal temperature for sulfuric acid production by the microbes. An agitator 314 (e.g., mixer, auger or the like) can agitate the solution, which agitation can be optimized for sulfuric acid production. The bioreactor 301 can optionally include one more substrates 318 for microbe attachment and culturing; however, it was found that the microbes do not need a substrate 318 and can float freely in the aqueous liquid in the bioreactor; however, the substrates are shown as floating/suspended particles or members (e.g., floating substrate 320) that can reside at any level, from top to bottom, of the aqueous liquid in the bioreactor. The substrates 318 can be any shape and any size and may be fixed to walls or the bottom or fixed to arms 322 extending into the liquid or floating. The microbes can be distributed, floating, or suspended anywhere in the liquid and/or with the substrates. A smart valve 11 may also be on the sulfuric acid output so that the desired or minimum pH level (e.g., maximum acidity) is obtained before output of sulfuric acid is achieved. The sulfuric acid output may also be based on mass fraction of H₂SO₄, density, or concentration (mol/L). In the figures, the relative location of an inlet or outlet is not limiting, and the inlet and outlets may be anywhere that is possible or feasible, unless a specific location for an inlet or outlet is specifically recited. Animal waste gas inlet 324, other gas inlet 326, SH₂ gas inlet 328, and SO₂ gas inlet 330 may also be included. Microbial waste outlet 332 may also be included.

The pH difference between sulfuric and sulfurous acids are shown below:

| | | |
|---|---|---|
| Sulfuric | N | 0.3 |
| Sulfuric | 0.1N | 1.2 |
| Sulfuric | 0.01N | 2.1 |
| Sulfurous | 0.1N | 1.5 |

FIG. 4 illustrates a bioreactor 401 configured for producing ammonium sulfate. Bioreactor 401 can include the same features and components of bioreactor 301 of FIG. 3. The ammonium sulfate bioreactor 401 can be configured as shown. In reference to FIGS. 1 and 2, the treated acidic water from outlet 156 or 264 can be sulfurous acid as described herein, which can be provided to the ammonium sulfate bioreactor 401 along with an ammonia source at ammonia inlet 410. Sulfuric acid may also be introduced at the sulfuric acid inlet 412, such as from the sulfuric acid collection tank 2a of FIG. 2 or from the tank 130 (when configured and operated as a bioreactor) via outlet 156. That is, the ammonium sulfate can be produced with any sulfuric acid, such as the sulfuric acid previously produced with microbes (e.g., sulfuric acid inlet). However, the ammonium sulfate bioreactor 401 is configured to generate the sulfuric acid from the microbes processing the sulfurous acid into sulfuric acid, which sulfuric acid is then used to produce ammonium sulfate with the ammonia/ammonium introduced therein. The ammonia or ammonium can be provided as ammonia, excrement (excrement inlet 414), compost (compost inlet 416), manure (manure inlet 418), urine (urine inlet 420), leachate (leachate inlet 422), and/or digestate (digestate inlet 424) which have an ammonia or ammonium content. The ammonia or ammonium and sulfuric acid can combine into ammonium sulfate mineral (NH₄)₂SO₄ which can precipitate. Other features of the ammonium sulfate bioreactor 401 can be the same as the bioreactor 301 that produces sulfuric acid because such production can be used for the ammonium sulfate generation when in the presence of ammonia or ammonium. The ammonium sulfate bioreactor 401 can also include a precipitate valve 430 at the bottom that can be selectively opened or closed to allow ammonium sulfate precipitate to be collected and provided to the ammonium sulfate outlet 433. The precipitate valve 430 can have a screen or any other porous member that allows the ammonium sulfate precipitate to fall therethrough, but which can retain the excrement, compost, or feces in the bioreaction chamber, or which can retain the substrates therein in the bioreaction chamber. The precipitate valve 430 can be opened to allow the ammonium sulfate precipitate to pass into the ammonium sulfate collector, which also allows for the contents of the bioreactor chamber to also pass into the ammonium sulfate collector 432, which contents can be liquid that includes sulfuric acid, sulfurous acid, ammonia, substrate, microbes, excrement, compost, feces, urine, or anything else in the bioreactor chamber. As such, the ammonium sulfate collector 432 can be sealed by closing the precipitate valve 430, and the liquid in the ammonium sulfate collector 432 can be recycled back to the bioreactor chamber via the valve 434 opening and pumping with the pump 436 through the liquid recycle pipe 438. When the liquid is removed, the ammonium sulfate can be collected. Otherwise, any methodology can be used to get the ammonium sulfate from the bioreactor 401. This bioreactor 401 can be used to prepare other minerals as described herein when the bioreactor includes the elements or substances to react to form the mineral. The minerals are described herein.

The bioreactor 401 can include an ammonia inlet 410 and/or ammonium inlet 410a in order to make ammonium sulfate or nitrate. The ammonia or ammonium can be from any source, synthetic or natural or organic. The ammonia or ammonium can be obtained from any barns or any other animal housing units, whether scrubbed (e.g., gases removed) or un-scrubbed (e.g., gases not removed), or from the animal waste therefrom. In some instances, gases obtained from scrubbing can be used as an ammonia source, which can include scrubbed gases from barns or any other animal housing units, or from the animal waste therefrom.

While ammonia sources are described herein, the source may also provide ammonium that can be used to produce the nitrogen containing acids and mineral salts described herein. As such, reference to ammonia sources may also include ammonium sources, and the presence of ammonia may also indicate a presence of ammonium. The ammonia may convert to ammonium, or the ammonium may convert to ammonia in the processes described herein for producing nitrogen containing acids and mineral salts.

Additionally, the other gases (other gas inlet 326) may be introduced into the ammonium sulfate reactor, such as carbon monoxide, carbon dioxide, H2S, hydrogen, or any others. Such gas streams may facilitate the microbe community that includes the SP microbes. That is, other microbes may contribute to the culture that supports the sulfuric acid producing microbes (SAP microbes), and these microbes may utilize other gases or any other food 426 Examples can include microbes that process nitrogen. Accordingly, the other gases or food may be used to promote a complex combination of different microbes for the formation of sulfuric acid. Such other gases and food may also be provided to the sulfuric acid producing bioreactor (e.g., FIG. 3). It should be recognized that for any of the bioreactors, the inputs can be separate and individual inputs (e.g., streams) or they can be combined in part (e.g., some combined), or all the inputs can be combined (e.g., combined into a common input stream).

The produced ammonium sulfate can be considered to be organic ammonium sulfate because the reagents for making the same can be organic. For example, the sulfurous acid supply can be from an organically certified sulfurous acid producing system. The ammonia can also be from an organic source, such as excrement, compost, feces (manure), or urine. Moreover, the use of microbes to produce the sulfuric acid can be considered an organic process since it is biological in nature. The combination of MPSA and ammonia produces the ammonium sulfate under ambient conditions. Thus, the process for producing ammonium sulfate may be considered to be organic.

In one option, the ammonium sulfate bioreactor can receive sulfur dioxide gas as the sulfur input that the microbes can then process into sulfuric acid. Accordingly, any sulfur dioxide gas or aqueous sulfur dioxide can be used for production of sulfuric acid and/or ammonium sulfate as described herein. Such sulfur dioxide gas or aqueous sulfur dioxide can be from the sulfur burner system shown herein and piped into the bioreactors.

In one aspect, a bioreactor 401a configured as described herein may use a phosphate input (phosphate/phosphorous inlet 464) in addition to or instead of ammonia to produce phosphate salt (phosphate salt outlet 460), such as in FIG. 4A. While phosphate input is shown, any phosphorous material that can be processed, such as with sulfuric acid, into a phosphate salt can be used. This can put the phosphate in proximity with the sulfuric acid to produce a phosphate salt. That is, any phosphate or phosphorous material can be introduced and processed to produce phosphate salt. Traditionally, phosphate fertilizers are obtained by extraction from minerals containing the anion PO43-. Now, soluble salts that include phosphate or any other phosphorous can be produced by chemical treatment of phosphate minerals with MPSA as produced herein. As such, phosphate containing material or minerals can be added to a bioreactor to make the phosphate salt with the MPSA. The most popular phosphate-containing minerals are referred to collectively as phosphate rock (phosphate rock inlet 462). The main minerals are Fluorapatite Ca5(PO4)3F (CFA) and hydroxyapatite Ca5(PO4)3OH. These minerals are converted to water-soluble phosphate salts by treatment with sulfuric acid.

Additionally, other mineral salts can be prepared with the MPSA, such as mineral salts that contain potassium, nitrogen, calcium, magnesium, manganese, iron, or any other element in a mineral salt. As such, reagents (e.g., minerals or rocks containing the desired element) for preparing mineral salts can be introduced into the bioreactor, such as shown in FIG. 4B, or alternatively MPSA/MPNA may be sprayed onto the reagents that are stacked or piled on a pad or in a tank to allow the MPSA or MPNA to contact it. The mineral inlet 464b and rock inlet 462b can provide the minerals. Some potassium minerals that can be used in the process or formed in the bioreactor (e.g., SO4) to produce the potassium mineral salts are shown in the table below. The potassium can be from any source. The elements to make the mineral salt may also be obtained from the excrement, compost, feces, or urine, which can contain a lot of different substances, such as N, P, K or any other micro nutrients. The mineral salt collector 432b can collect the mineral salt that is provided out the mineral salt outlet 460b.

| | | K₂O content |
|---|---|---|
| Mineral | Composition | (approx %) |
| Chlorides: | | |
| Sylvinite | KCl•NaCl | 28 |
| Sylvite | KCl | 63 |
| Carnalite | KCl•MgCl₂•6H₂O | 17 |
| Kainite | 4KCl•4MgSO₄•11H₂O | 18 |
| Sulfates: | | |
| Polyhalite | K₂SO₄•2MgSO₄•2CaSO₄•2H₂O | 15 |
| Langbeinite | K₂SO₄•2MgSO₄ | 22 |
| Schoenite | K₂SO₄•MgSO₄•4H₂O | 23 |
| Nitrates: | | |
| Niter | KNO₃ | 46 |

In one aspect, compost or manure can be leached with water, or by MPSA or MPNA or any other microbially produced acid, or by sulfurous acid or, to facilitate production of the mineral salts.

The main micronutrients that can be in the mineral salts produced in the bioreactors can include sources of iron, manganese, molybdenum, zinc, and copper. As for the macronutrients, these elements are provided as water-soluble salts. Iron presents special problems because it converts to insoluble (bio-unavailable) compounds at moderate soil pH and phosphate concentrations. For this reason, iron is often administered as a chelate complex, e.g., the EDTA derivative. Any micronutrient can precipitate in the bioreactors with the mineral salts.

The mineral salts made in the bioreactors can be combined to make the multi-nutrient salts, or the multi-nutrient salts can be made in the same bioreactor by introducing and reacting the appropriate reagents.

In one aspect, the sulfurous acid or sulfur dioxide gas can be bubbled up through the bioreactor chamber in order to contact the microbes that produce the MPSA.

Additionally, any of the input streams shown for the figures, such as FIGS. 4-4B or any bioreactor, can be supplied into the bioreactor alone, or any of the input streams can be combined or all of them can be combined. In one example, input streams that do not chemically react with each other can be combined into a common input and pumped into the bioreactor. This allows input as single components or in poly-component forms.

(NH4)2SO4, is an inorganic salt with a number of commercial uses. The most common use is as a soil fertilizer. It contains 21% nitrogen and 24% sulfur. For example, the ammonium sulfate fertilizer contains 21.4% N. Laboratory measurements made on the cultured ammonium sulfate produced by the system described herein have assayed as high as 24% nitrogen. As such, the system can produce super nitrogen ammonium sulfate having above 21.4% nitrogen, or above 24% nitrogen. Even higher percentages of nitrogen may also be possible by manipulating the reaction conditions. It is thought, without being bound thereto, that the excess ammonia allows for nitrogen (e.g., ammonia) to be trapped in the lattice of the precipitated ammonium sulfate to produce super nitrogen ammonium sulfate. Any reference to nitrogen therein is understood to be part of a substance, such as nitrogen gas (N2) or ammonia, or ammonium or nitrite or nitrate or nitric acid or other. Any of the mineral salts produced herein can be used for fertilizer or other use for the minerals.

The ammonium sulfate bioreactor may also be used to reduce ammonia pollution by consuming the ammonia in animal excrement (e.g., chicken excrement). This can help a farm smell better. In one aspect, the ammonia can be removed by processing as described herein. The ammonia can be removed from compost or animal housing structures. Additional odors emanating from manure or animal housing can also be reduced further by use of a bioreactor to neutralize VOC's and other compounds once the ammonia in the air has been minimized by the methods described herein.

In one aspect, the tank 130 of the systems described herein can be configured as an ammonium sulfate bioreactor or a bioreactor for any of the mineral salts described herein when it includes an ammonia or other nitrogen input. The ammonia input can be the same as described herein, such as excrement, compost, feces, and/or urine. This can allow for the ammonium sulfate to be produced at the same time the MPSA is produced. The ammonium sulfate produced in tank 130 may then be collected.

In one aspect, the production of sulfuric acid and/or ammonium sulfate can be configured as a countercurrent process, which can be batch or continuous. The countercurrent process can include opposing flows or one flow passing through a more stationary flow. For MPSA production, the sulfurous acid can be flowed through the aqueous media having the sulfuric acid producing microbes. This can be accomplished by piping the MPSA into the bioreactor chamber with a flow rate while the aqueous media and microbes are either stationary, in an opposing flow, or a circular flow (e.g., by vortex stirring), where the MPSA flow is in a direction different from the aqueous media flow. In one option, the sulfur dioxide gas can be bubbled from the bottom up through the bioreactor, which can cause formation of the sulfurous acid and then the MPSA. This configuration may be applied to the other bioreactors to produce other mineral salts.

For ammonium sulfate production, the nitrogen and/or sulfur dioxide can be bubbled from the bottom up through the media having the microbes. Alternatively, any other countercurrent operation can be used, such as by piping with flow rates the ammonia reagent and the sulfuric acid reagent past each other or in different directions, such as either flow being stationary, flows in an opposing flow, or one flow in a relative circular flow (e.g., by vortex stirring). The formation of ammonium sulfate then causes precipitation.

In one example, the sulfurous acid flow is flowing in from the bioreactor, while the ammonia is bubbling up from the bottom, such that the ammonia is coming through the media populated with microbes, and the gas that leaves from the top vent is stripped of most of the ammonia. This allows gas to vent from the bioreactor with less smell. Also, after the ammonium sulfate starts precipitating it can be withdrawn from the bioreactor in any way. In one example, the fluid/liquid is removed from the tank, and any solid ammonium sulfate is obtained from the fluid/liquid. The fluid/liquid can be filtered and centrifuged, where the centrifuge spins out the water from the ammonium sulfate, and the remaining fluid/liquid can be recycled to the bioreactor. The ammonium sulfate can be collected after the water is spun out via centrifugation. The sulfurous acid can be recycled back to the sulfur burner system, and/or recycled back to the bioreactor. Also, the use of animal waste allows for many different microbes in the bioreactor along with the microbes that produce sulfuric acid. The gas vent may also be recycled into an inlet, such as at the bottom of the bioreactor.

Some methods of obtaining the mineral salts produced in the bioreactors can include removing the liquid from the mineral salt solution. The removal of liquid from the mineral salt solution can include: using an reverse osmosis (RO) to remove the water; using a membrane or filter to filter the mineral salt out for collection; using pervaporation (e.g., a processing method for the separation of mixtures of liquids by partial vaporization through a non-porous or porous membrane); evaporation of water; distillation; a cryogenic process, lyophilization; using hydrocyclones; using evaporators; using crystallizers, and using thermal energy to remove water from the mineral salt; or any other. In one example, heat from sulfur burners and/or composting (e.g., as described herein) can be used to concentrate the mineral or remove the water from the mineral salt.

The ammonia supply for the preparation of mineral salt can be from a compost, such as compositing animal and/or other wastes. The composting can in one example be by the Berkley Technique, which is a rapid composting system that can completely compost in about 2-3 weeks (e.g., 17 days). The composting material is put into a large bin or pile in such a way that here is airflow up from the bottom through the compost, which distributes oxygen throughout the compost. The composting uses aerobic microbes that use the oxygen. The compost can heat to a high temperature, such as 160-170 degrees F. Water can be introduced to the compost, such as for temperature management and/or mineral leaching (if the leachate is captured and introduced to the system). Certain adjustments can be made to the manure, such as elevating pH or reducing the preferred carbon:nitrogen ratio, or the addition of extra microbes such as EM-1 to increase ammonia production and/or release during the composting process. Any ammonia released from the compost can be collected and piped into an ammonium sulfate bioreactor. In one aspect, a grain silo can be used for such composting, such as a silo that is 50+ feet in diameter, with or without stirrers/augers.

In composting, the preferred animal waste in terms of nitrogen content is: poultry, swine, dairy cows, and then field cattle; however, fish waste (e.g., pisci-waste) may also be used. The pisci-waste may also be used as a source for nitric acid. In one aspect, the pisci-waste can be introduced into the bioreactors described herein. The pisci-waste may also be used in the composting silos. In one example, the pisci-waste can be obtained as an aqueous solution (e.g., from fish farm or aquarium or other source) and then introduced into the bioreactors described herein. The pisci-waste may be especially useful for the bioreactors that make nitrite, nitrate, or nitric acid.

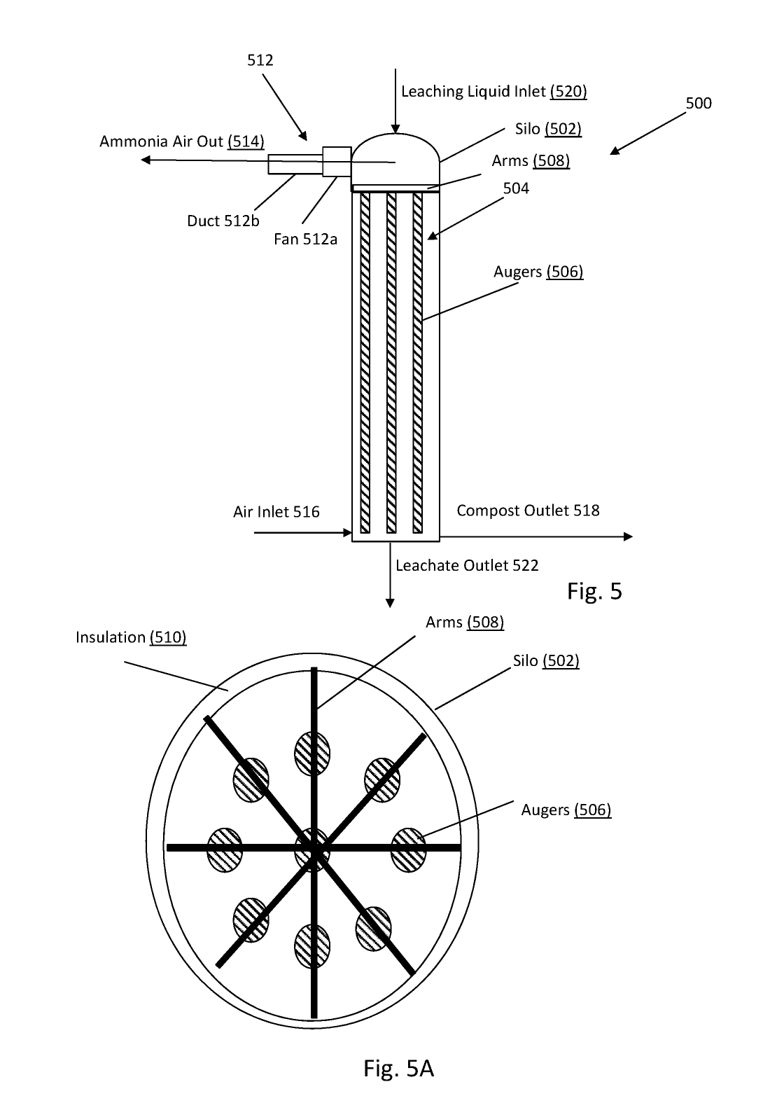

FIGS. 5-5A illustrates a composting system 500 having a silo 500 and stirring system 504 that may be used for large scale application of the Berkley Technique for composting. The silo 502 may include the stirring system 504 with one or more augers 506 that go down through the silo, which augers 506 can be central or eccentric. The silo 502 has augers 506 that go down through the pile of the compost, and the augers 506 can pull the lower compost up and cycle the higher compost back down. The augers 506 can be located on arms 508 that can be eccentric to move the augers 506 in a mixing pattern, which is used to constantly create new air pathways and homogenize the compost. The air can be pulled from an air inlet 516. The mixing of the compost in the silo 502 keeps the temperature uniform to move hot internal compost to the outside where it is cooler, and vice versa. The wall can be insulated with insulation 510, such as a metal wall wrapped with spray foam or any other insulation. The inner wall may be coated to protect the wall from metal corrosion. The silo 502 configured as a composter allows the compost to heat up quickly, bring air in the bottom and pull ammonia air out the top (ammonia air out 514). The ammonia containing gases can be captured with a captivating fan 512a and duct 512b system 512, which can then be piped to the ammonium sulfate bioreactor (e.g., ammonia inlet). This allows for the air around the silo 502 to have reduced ammonia smell. The composting technique can reduce the composting time to as little as 5-17 days. The goal of composting can be to remove as much ammonia or any other nitrogen source as quickly and thoroughly as possible, with or without fully composting the manure. It is noted that the arms are optional as the augers 506 can be stationary. In one aspect, the silo 502 can be devoid of augers or any other mixer. In one aspect, the augers 506 shown in the figure are replaced by vented conduits that can provide oxygen to the internal portions of the silo. The vented conduits can include mesh or any other semi-permeable layer (e.g., membrane) that keeps the compost out of the vented conduits but allows gases, such as oxygen, to exchange between compost material and conduit. The silo 502 may be operated create some regions (e.g., pockets in the compost) where anaerobic processes occur alongside of aerobic processes. The combination of anaerobic processes occur alongside of aerobic processes may occur at least temporarily or may be sustained. For example, air passing through the augers 506 may have aerobic processes in the adjacent composting material, while internal areas not near an air supply may have anaerobic processes.

For example, the type of stirring system 504 that can include the arms 508 and augers 506 that can be used, such as for example, one arm 508 with one or more augers 506, where the arm 508 can rotate around the silo 502. In one option, the augers 506 can move back and forth along the arm 508, such as where the arm 508 provides a track for the augers 506 to move laterally across the silo 502 as the arms 508 are stationary or rotating around the silo 502. The stirring system 504 can mix the driest compost at the bottom of the silo 502 with the wetter compost toward the top. This results in more uniform moisture content. Stirring loosens the compost, reduces static pressure and increases airflow, which allows for the compost to dry more quickly and efficiently. Stirring also breaks up the “hotspots.” In one example, the silo 502 can include the FASTSTIR™ stirring system by SUKUP™.

The used compost can be moved out the compost outlet 518. Also, a leaching liquid that can leach minerals and substances can be provided in a leaching liquid inlet 520, such that the leachate having the minerals and substances can be withdrawn out the leachate outlet 522.

In one aspect of the methodologies described herein, the microbes can be obtained from a geothermal area and selected to obtain microbes that make MPSA from gaseous/aqueous sources of sulfur including H2S, SO2, SO3, HSO3, sulfurous acid, or any other liquid or gas sulfur source, optional solid sulfur. The MPSA can then be used to make Mascagnite or ammonium sulfate compounds from any source of ammonia gas, such as from manure piles, composting manure, animal barns/housing, or any other natural sources of gaseous or liquid ammonia. While manure, feces, excrement, urine or any other animal waste can be used for the source of ammonia or ammonium or any other reagent (e.g., elements or minerals), however, such recitations may include the solid, liquid or gaseous emissions therefrom. For example, the manure, feces, excrement, urine or any other animal waste used in the process can be the gases obtained, generated, or derived therefrom. For example, the manure, feces, excrement, urine or any other animal waste can be in an enclosed housing (e.g., barn, composter or composing silo as described herein) and gases and leachates extracted or withdrawn from such an enclosed housing can be used in the processes described herein, such as use in the bioreactors.

In one embodiment, the processes described herein can use sewer sludge or other animal waste sludge to make the microbial acids and/or mineral salts. However, such use of sewer sludge may include contaminants (e.g., heavy metals, pharmaceuticals and their byproducts, etc.) that do not allow for organic certification of the microbial acid or mineral salt. In any event, sewer sludge or other contaminated animal waste can be used to prepare microbial acids or mineral salts as described herein.

The methodologies described herein can be used with the technologies of U.S. 2014/0170725 and/or U.S. 2014/0017161, which are incorporated herein by specific reference. That is, the technologies of these publications can be modified with the technologies described herein, which can be used for making MPSA and mascagnite with MPSA producing microbes.

In one aspect, the methods described herein can be performed without the use of naturally occurring gypsum, barite, and sulfur.

The microbes can be obtained from the Iron Mountain superfund site, or from contaminated water therefrom. Also, the microbes can be obtained from any geothermal hot spot or water therefrom.

In one embodiment, the MPSA can be made using gaseous sulfur dioxide. This can be for MPSA or ammonium sulfate. The system described herein can burn sulfur, and immediately thereafter introduce the SO2 gas stream into the microbe populated reactor tank (e.g., 130) or any of the bioreactors as a gas. That is, the sulfur dioxide can be used for MPSA production without first being converted into H2SO3 acid. This can allow for a minimalistic system of a sulfur burner having a burn pot and piping with optionally a fan that pipes the sulfur dioxide into the bioreactor.

The MPSA as described herein can be performed on location, such as at a farm, and the MPSA can be used for any purpose. The processing can be tailored to modulate the pH of the MPSA for different uses. As such, the microbes can be exposed to various pH levels to obtain microbes capable of generating MPSA while being in a highly acidic environment.

In one embodiment, the processes described herein include taking the microbes from their natural habitat, and placing them in a non-natural habitat to produce sulfuric acid. One example is to put the cultivated microbes in a bioreactor in the presence of MPSA and ammonia from the waste of farm animals. In one aspect, the microbes can be obtained from acid mine drainage where the microbes attack sulfides in dump material or in the mines and then the microbes produce MPSA. The microbes may be from a location, such as Kawha Ljen (e.g., volcanic crater lake in Indonesia), and such microbes therefrom may facilitate the production of ammonium sulfate.

In one embodiment, the methodologies described herein can facilitate the production of MPSA, where the sulfur source for the microbes is gaseous, aqueous, or liquid. That is, MPSA can be produced. Such MPSA can be used to produce ammonium sulfate. However, any MPSA may be used to produce ammonium sulfate in order to obtain this mineral salt. Also, any natural source of MPSA (e.g., Kawha Ljen, Iron Mountain, or any volcanic crater lake, or any naturally occurring source of MPSA or mixtures of MPSA with hydrochloric acid or any other naturally occurring acids, etc.) can be used with any natural source of ammonia to produce the ammonium sulfate or any other mineral salts as described herein.

See also U.S. 2008/0102514, which can be modified with the technology described herein, and vice versa.

In one embodiment, the microbes can be from any mineral processing plant waste streams, or any tailings, mine dump or the like. For instance, coal wash plants separate pyrite and other sulfur compounds from high sulfur coal. The sulfur containing reject material is stored in plant waste piles and/or tailings, and over time usually start releasing H2SO4 due to the action of microbes. These microbes can be obtained for use as described herein. Also, coal mine dumps may contain poor quality coals and/or carboniferous shales with pyrite or other sulfur compounds material mined with the coal. These mine dumps can also be used to cultivate the microbes, and the microbes can be obtained and selected as described herein.

In one embodiment, the methods can include burning sulfur, sulfides, or sulfur containing compounds, such as coal, to make sulfur dioxide to use as described herein.

Many species of nitrifying bacteria have complex internal membrane systems that are the location for key enzymes in nitrification: ammonia monooxygenase which oxidizes ammonia to hydroxylamine, and nitrite oxidoreductase, which oxidizes nitrite to nitrate. Nitrification in nature is a two-step oxidation process of ammonium (NH4+) or ammonia (NH3) to nitrate (NO3−) catalyzed by two ubiquitous bacterial groups. The first reaction is oxidation of ammonia or ammonium to nitrite by ammonia or ammonium oxidizing bacteria (AOB) represented by the “Nitrosomonas” species. The action may also be done with ammonia, especially in the presence of hydrogen atoms. The process may also include changing ammonia to ammonium. The second reaction is oxidation of nitrite (NO2−) to nitrate by nitrite-oxidizing bacteria (NOB), represented by the Nitrobacter species. Ammonia oxidation in autotrophic nitrification is a complex process that requires several enzymes, proteins and presence of oxygen. The key enzymes, necessary to obtaining energy during oxidation of ammonium to nitrite are ammonia monooxygenase (AMO) and hydroxylamine oxidoreductase (HAO). A transmembrane copper protein catalyzes the oxidation of ammonia or ammonium to hydroxylamine taking two electrons directly from the quinone pool. This reaction requires O2. In the second step, a trimeric multi-heme c-type HAO converts hydroxylamine into nitrite in the periplasm with production of four electrons. The stream of four electron are channeled through cytochrome c554 to a membrane-bound cytochrome c552. Two of the electrons are routed back to AMO, where they are used for the oxidation of ammonia (quinol pool). Two electrons are used to generate a proton motive force and reduce NAD(P) through reverse electron transport. Nitrite produced in the first step of autotrophic nitrification is oxidized to nitrate by nitrite oxidoreductase (NXR). It is a membrane-associated iron-sulfur molybdoprotein, and is part of an electron transfer chain which channels electrons from nitrite to molecular oxygen. The nitrifying bacteria that oxidize ammonia into nitrate include Nitrosotalea, Nitrosomonas, Nitrosococcus, and Nitrosospira, and likely others (e.g., nitrite producing microbes). The nitrifying bacteria that oxidize nitrite into nitrate include Nitrobacter, Nitospina, Nitrococcus, and Nitrospira, and likely others (e.g., nitrate producing microbes). However, these classifications are not limiting, and the different nitrifying bacteria may be present in the production of nitrite and/or nitrate.

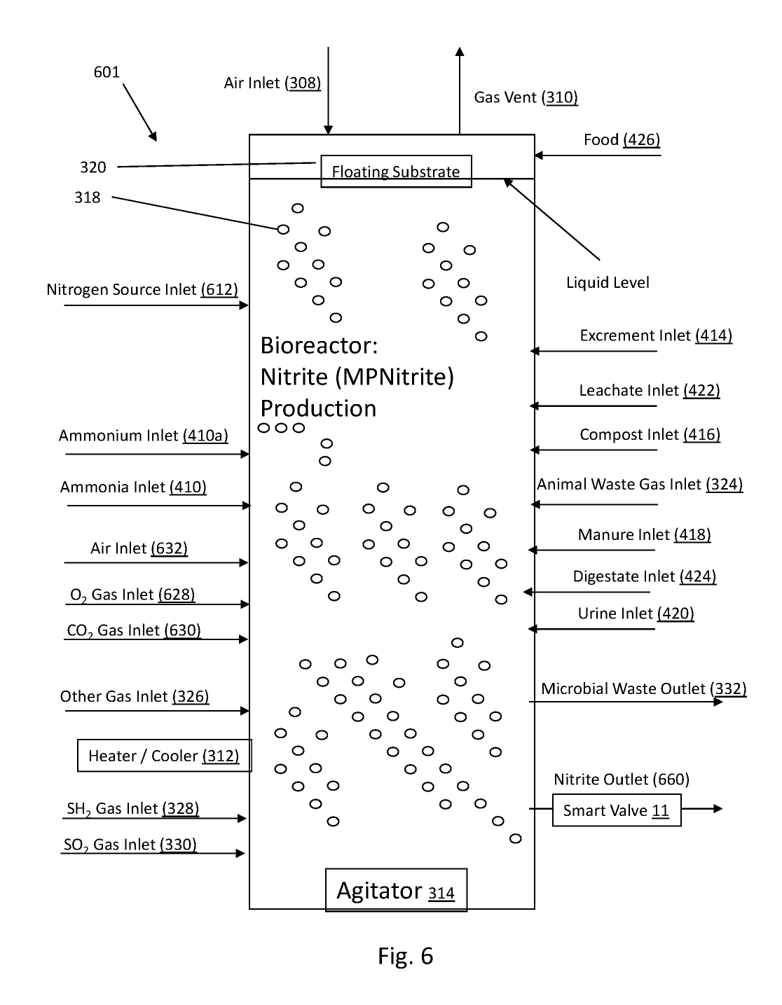

FIG. 6 shows a bioreactor 601 that can be used in a process of using microbes for making nitrite (NO2), which can be referred to as microbially-produced nitrite (e.g., MPNitrite). The bioreactor 601 can include inlets, outlets, and components as in any of the other bioreactors described herein. In one example, the processes can be performed by capturing and using streams of ammonia and/or ammonium and/or CO2 emitted from a compost pile or barn or ammonia/ammonium scrubber or any other appropriate source, and mix the captured streams with water and nitrifying microbes (e.g., Nitrosotalea, Nitrosomonas, Nitrosococcus, Nitrosospira, or any other microbe that produces nitrite) in order to produce nitrites. As shown, the bioreactor can be configured as described herein, and include the nitrifying microbes in addition to any sulfuric acid producing microbes or in place of them. In one aspect, such a nitrite producing bioreactor 601 can be devoid of the sulfuric acid producing microbes. The nitrifying microbes can be suspended with or without a substrate 318, or may be on a floating substrate 320 or substrate attached to the bioreactor. In order to make nitrite, the bioreactor 601 may include other microbes that facilitate a culture of nitrifying microbes. It should be recognized that NO2 can be nitrogen dioxide, which may be nitrite ion or nitronium ion as well as their salts. As such, reference to nitrite is also a reference to any salt thereof, which can be determined by the presence of suitable counter ions to make the nitrate salt. Accordingly, the bioreactor 601 can include a nitrogen source inlet 612, air inlet 632, oxygen gas inlet 628, carbon dioxide gas inlet 630, and nitrite outlet 660.

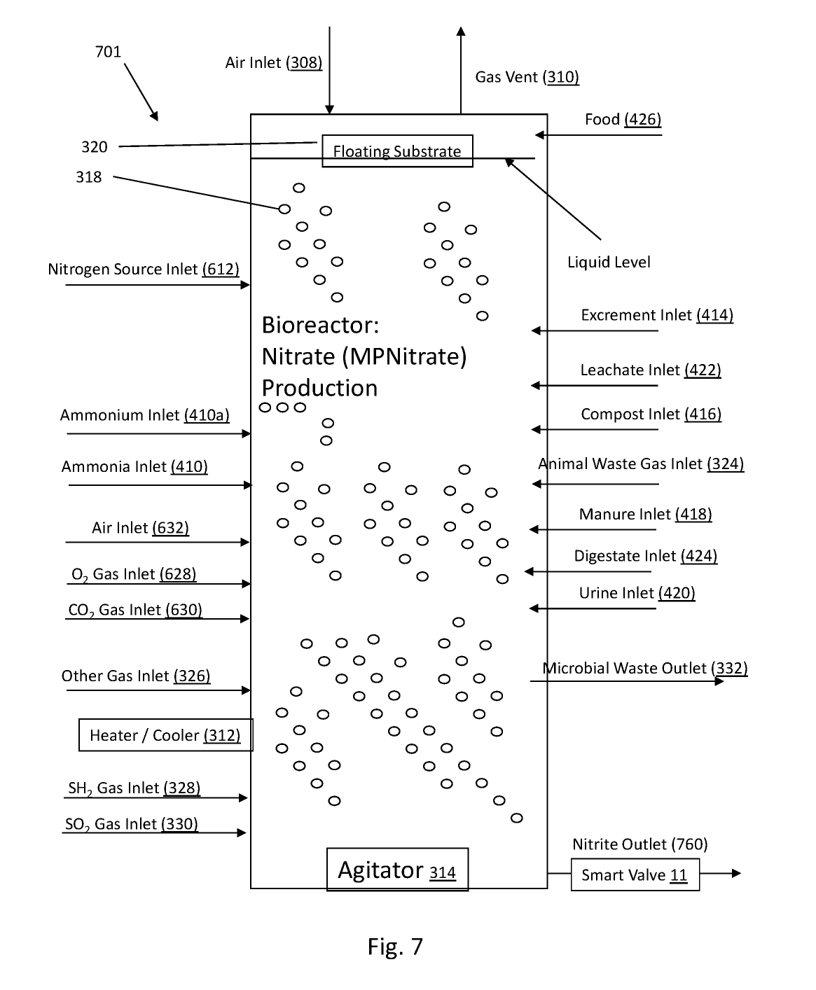

FIG. 7 shows a bioreactor 701 that can be used in a process of using microbes for making nitrate (NO3), which can be referred to as microbially-produced nitrate (e.g., MPNitrate). In one example, a source of nitrite (e.g., nitrogen source inlet 612) can be provided that can be used to produce the MPNitrate. In an example, the nitrite can be MPNitrite produced such as shown with the bioreactor of FIG. 6. As such, the nitrite can be converted to nitrate with the microbes. In one example, the processes can be performed by capturing and using streams of ammonia and/or ammonium and/or CO2 emitted from a compost pile or barn or ammonia/ammonium scrubber or any other appropriate source, and mix the captured streams with water and nitrifying microbes (e.g., Nitrosotalea, Nitrosomonas, Nitrosococcus, Nitrosospira, or any other microbe that produces nitrite) to produce nitrite that is then processed into nitrate by nitrifying microbes (e.g., Nitrobacter, Nitrococcus, Nitrospina, Nitrospira, or any other microbe that produces nitrates) in order to produce nitrites and then to produce nitrates. As shown, the bioreactor 701 can be configured as described herein, and include the nitrifying microbes in addition to any sulfuric acid producing microbes or in place of them. In one aspect, such a nitrate producing bioreactor 701 can be devoid of the sulfuric acid producing microbes. The nitrifying microbes can be suspended with or without a substrate 318, or may be on a floating substrate 320 or substrate attached to the bioreactor. In order to make nitrate, the bioreactor 701 may include other microbes that facilitate a culture of nitrifying microbes. The production of nitrate can refer to the production of any nitrate salt, which can be determined by the presence of counter ions to make the nitrate salt. While not specifically shown, the nitrate can be extracted as a liquid (e.g., when ionized) or as a solid, such as when a salt. Accordingly, the bioreactor 701 can include a nitrogen source inlet 612 (e.g., nitrite), oxygen gas inlet 628, carbon dioxide gas inlet 630, air inlet 632, and nitrate outlet 760.

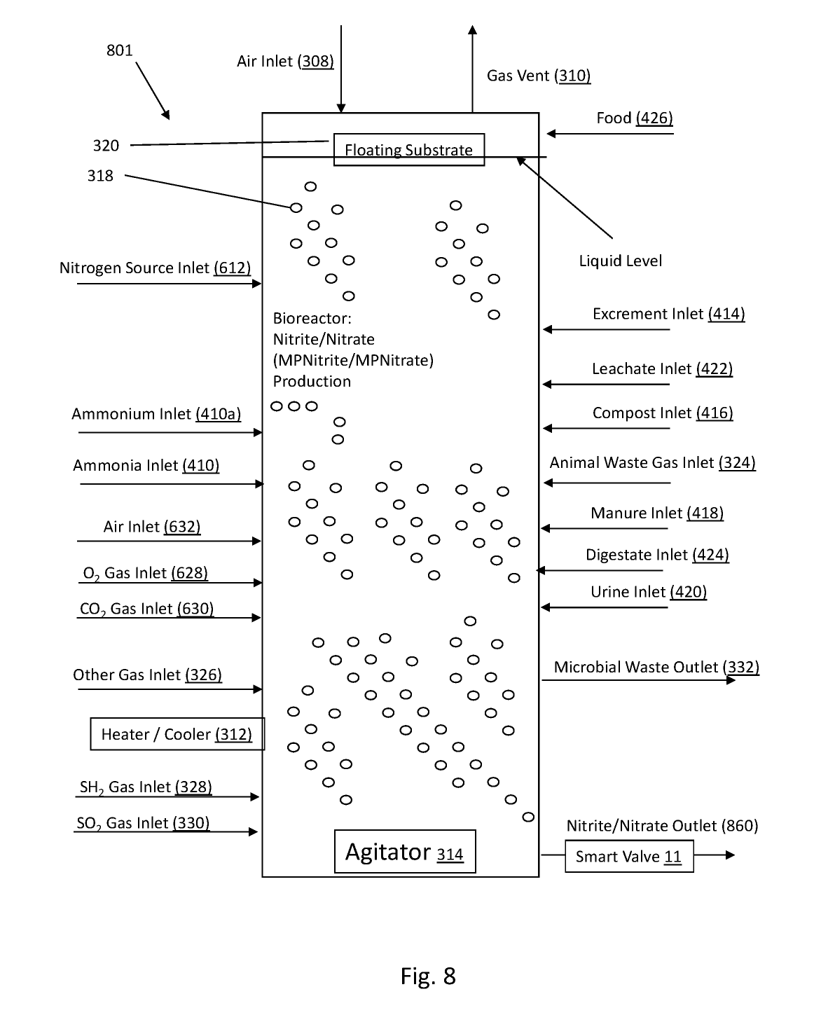

FIG. 8 shows a bioreactor 801 that can be used in a process of using microbes for making nitrite (NO2) and nitrate (NO3), which can be referred to as microbially-produced nitrite (e.g., MPNitrite) and microbially-produced nitrate (e.g., MPNitrate). In one example, the processes can be performed by capturing and using streams of ammonia and/or ammonium and/or CO2 emitted from a compost pile or barn or ammonia/ammonium scrubber or any other appropriate source, and mix the captured streams with water and nitrifying microbes (e.g., Nitrosotalea, Nitrosomonas, Nitrosococcus, Nitrobacter, Nitrococcus, Nitrosospira, Nitrospina, Nitrospira, or any other) in order to produce nitrites and or nitrates. As shown, the bioreactor can be configured as described herein, and include the nitrifying microbes in addition to any sulfuric acid producing microbes or in place of them. In one aspect, such a nitrite/nitrate producing bioreactor 801 can be devoid of the sulfuric acid producing microbes. The nitrifying microbes can be suspended with or without a substrate, or may be on a floating substrate or substrate attached to the bioreactor. In order to make nitrite and nitrate, the bioreactor may include other microbes that facilitate a culture of nitrifying microbes. Accordingly, the bioreactor 801 can include a nitrogen source inlet 612 (e.g., nitrite or other), oxygen gas inlet 628, carbon dioxide gas inlet 630, air inlet 632, and nitrite/nitrate outlet 860.

In one aspect, the nitrite and/or nitrate, such as MPNitrite and/or MPNitrate, can then be used to make nitric acid (HNO3) via any reaction or processing. This process can form microbially produced nitric acid (e.g., MPNA), such as when the MPNitrite and/or MPNitrate is converted to nitric acid. This MPNA can be used as the basis for even more concentrated forms of nitrogen based salts. Such a method can be used with any ammonia and/or carbon dioxide source, such as by using the processes described herein. In one aspect, the MPNitrate can by hydrogenated to MPNA.

Nitric acid can be made by reaction of nitrogen dioxide (NO2) with water to form nitric acid and nitric oxide (NO), and the NO produced by the reaction can be reoxidized by the oxygen in air to produce additional nitrogen dioxide that can be reacted with water to produce more nitric acid and nitric oxide (NO). Bubbling nitrogen dioxide through the bioreactor can help to improve nitric acid yield. Additionally, nitric acid can be made by reacting a nitrate (e.g., nitrate salt) with an acid (e.g., sulfuric acid or hydrochloric acid).

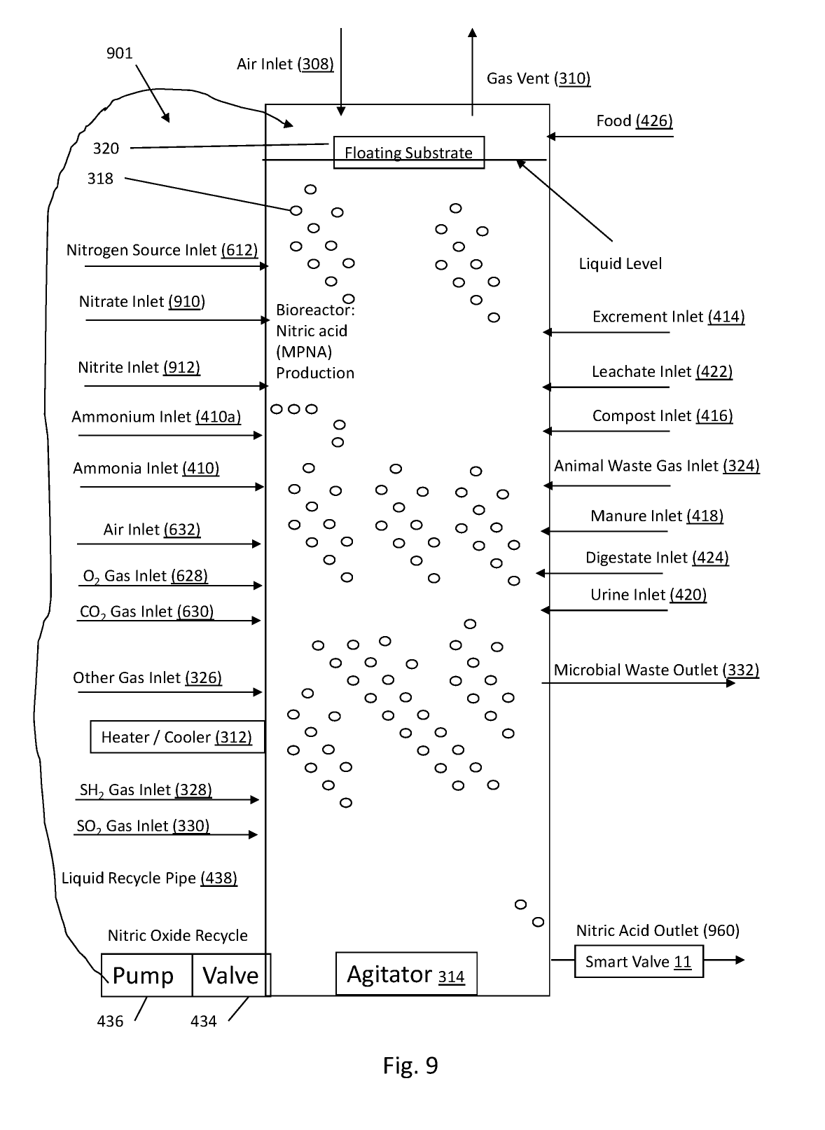

FIG. 9 shows a bioreactor 901 that can be used in a process of using microbes for making nitric acid from nitrite (e.g., MPNitrite) and/or nitrate (e.g., MPNitrate), which can be referred to as microbially-produced nitric acid (e.g., MPNA). Accordingly, a nitrate inlet 910 and nitrite inlet 912 can be provided, where the nitric acid outlet 960 provides the output. In one example, the processes can be performed by capturing and using streams of ammonia and/or ammonium and/or CO2 emitted from a compost pile or barn or ammonia/ammonium scrubber or any other appropriate source, and mix the captured streams with water and nitrifying microbes (e.g., Nitrosomonas, Nitrosococcus, Nitrobacter, Nitrococcus, Nitrosospira, Nitrospina, Nitrospira, or any other) in order to produce MPNitrite and/or MPNitrate. The MPNitrite and/or MPNitrate is then processed into nitric acid, such as by the presence of water or with the presence of another acid (e.g., hydrochloric acid or sulfuric acid or MPSA). In one option, MPNitrite is reacted with water to produce nitric acid, such as MPNA. In one option MPNitrate is reacted with acid to produce nitric acid, such as MPNA. As shown, the bioreactor can be configured as described herein, and include the nitrifying microbes in addition to any sulfuric acid producing microbes or in place of them. In one aspect, such a nitrite/nitrate producing bioreactor can be devoid of the sulfuric acid producing microbes. In one aspect, the nitric acid can be made in the same reactor that makes the MPNitrite and/or MPNitrate, such as described herein. The nitrifying microbes can be suspended with or without a substrate, or may be on a floating substrate or substrate attached to the bioreactor. In order to make nitrite and nitrate, the bioreactor may include other microbes that facilitate a culture of nitrifying microbes.

The bioreactors can also be used to make mineral salts such as ammonia nitrate. The preparation of ammonium nitrate can be by using a nitrate salt such from an MPNitrate salt, and ammonia. The process can use the same ammonia sources as described herein and the MPNitrate can result in ammonia nitrate. Here, the ammonia can be reacted with the nitrate in the presence of an acid (e.g., sulfuric acid, MPSA). Ammonium nitrate can also be produced by reacting nitric acid and ammonia. These reactions can be performed in the same type of bioreactors with the appropriate inputs and conditions. The ammonium nitrate can be prepared in the presence of the microbes, such as in a multi-stage reaction scheme in the bioreactor.

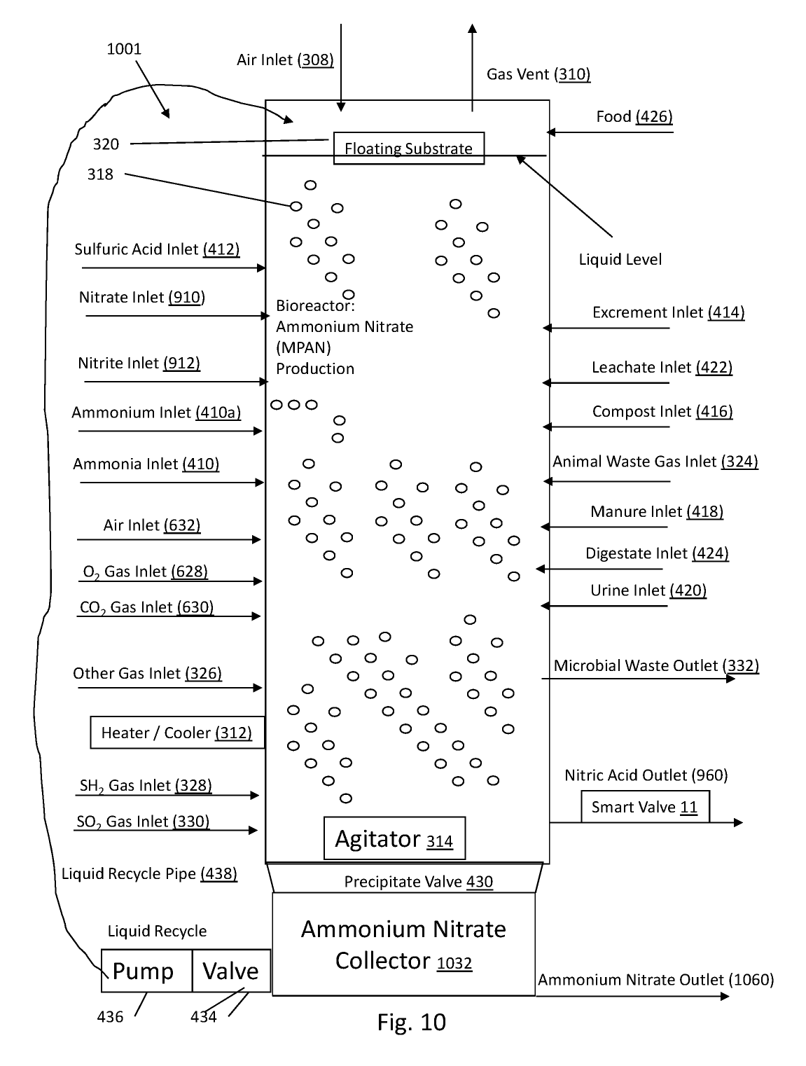

FIG. 10 shows a bioreactor 1001 that can be used in a process of using microbes for making ammonium nitrate. The bioreactor 1001 can include can include any of the components of other bioreactors, and may include the inputs and output as shown. The process can use microbes to make the nitrite (e.g., MPNitrite) to make MPNitrate that then can make ammonium nitrate to make ammonium nitrate or make nitric acid that can them make ammonium nitrate. Also, the process can use microbes to make MPNitrate, which can then be processed as described herein to obtain ammonium nitrate. Either process uses microbes, and thereby it which can be referred to as microbially-produced ammonium nitrate (e.g., MPAN). In one example, the processes can be performed by capturing and using streams of ammonia and/or ammonium and/or CO2 emitted from a compost pile or barn or ammonia/ammonium scrubber or any other appropriate source, and mix the captured streams with water and nitrifying microbes (e.g., Nitrosomonas, Nitrosococcus, Nitrobacter, Nitrococcus, Nitrosospira, Nitrospina, Nitrospira, or any other) in order to produce MPNitrite and/or MPNitrate. The MPNitrite and/or MPNitrate is then processed into ammonium nitrate. In one option, MPNitrite is reacted with water to produce nitric acid that is processed to produce the MPAN. In one option MPNitrate is reacted with acid to produce ammonium nitrate. As shown, the bioreactor 1001 can be configured as described herein, and include the nitrifying microbes in addition to any sulfuric acid producing microbes or in place of them. In one aspect, such a ammonium nitrate producing bioreactor 1001 can be devoid of the sulfuric acid producing microbes. In one aspect, the ammonium nitrate can be made in the same reactor that makes the MPNitrite and/or MPNitrate, such as described herein. The nitrifying microbes can be suspended with or without a substrate, or may be on a floating substrate or substrate attached to the bioreactor. In order to make nitrite and nitrate, the bioreactor may include other microbes that facilitate a culture of nitrifying microbes. The ammonium nitrate can be collected as a precipitate or as any way described herein with the ammonium sulfate precipitate. As such, the ammonium nitrate collector can be operated as the ammonium sulfate collector. The ammonium nitrate collector 1032 can collect the ammonium nitrite as with other mineral collectors described herein and provided out the ammonium nitrate outlet 1060.

Additionally, the bioreactors may include microbes that process nitrogen gas (e.g., N2), and the methods can include processing nitrogen gas with the microbes in the bioreactors. The nitrogen gas may be processed by Azotobacter or Clostridium or other microbes, such as processing nitrogen gas into NH2 (e.g., protein NH2) that can then be processed by other microbes. In one aspect, these microbes can process nitrogen gas from air or any nitrogen gas bubbled through the bioreactors. Additionally, microbes that produce ammonia can be included in the bioreactors and processes. Such ammonia producing microbes may include clostridia, enterobacteria, Bacillus, streptococci, and micrococci, or others.

Accordingly, the methods described herein using sulfur-processing microbes to produce sulfuric acid (MPSA) may be modified by using nitrogen-processing microbes to produce nitrites that then are hydrogenated to produce nitric acid (e.g., MPNA). The nitric acid can be a product, or it can be used in the formation of mineral salts. The changes in the bioreactors, reagents or input streams, or microbe populations in the bioreactors described herein (e.g., FIGS. 3, and 4-4A) can be made to use the nitrogen-processing microbes in addition to the sulfuric acid-producing microbes or to use the nitrogen-processing microbes instead of the sulfuric acid-producing microbes.

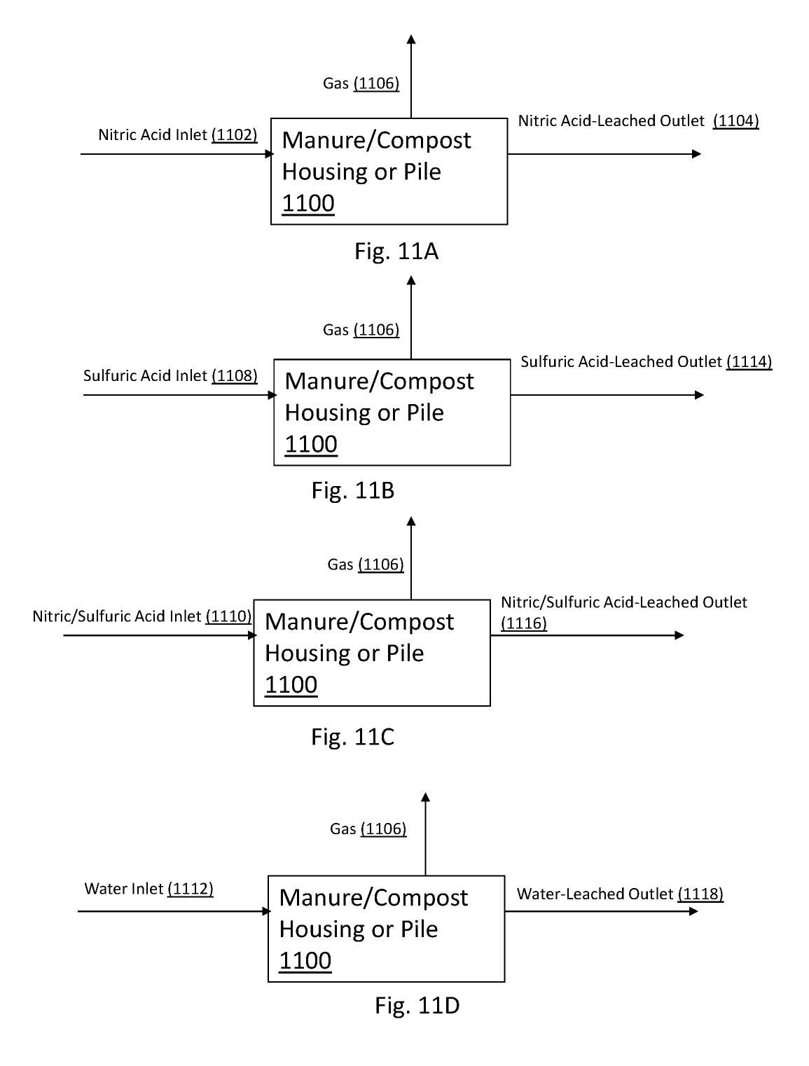

Additionally, the sulfuric acid and/or nitric acid can be used to process the manure, feces, excrement, urine or any other animal waste. Such processing can include facilitating any reaction between the acid and the animal waste. In one aspect, the reaction can facilitate generation or emission of gases (e.g., ammonia), which can be collected and supplied to the bioreactors. In another aspect, water or microbial produced acids can leach substances, such as elements or minerals, from the animal waste, and such leached output can be obtained from the waste. FIGS. 11A, 11B, and 11C show that gas can be emitted and the leached product (e.g., nitric acid-leached outlet 1104, sulfuric acid leached outlet 1114, nitric/sulfuric acid leached outlet 1116, or water-leached outlet 1118). The leached product may include counter ions for preparing fertilizers, such as K, P, Ca, Mg, or any other. The leached product may also include minerals that may be present in the animal waste. The leached products can then be used to prepare mineral salts, such as those described herein.

FIG. 11A shows the nitric acid inlet 1102 into the manure/compost housing or pile 1110 results in the nitric acid-leached product provided out the outlet 1104, and where gas is released out the gas outlet 1106. FIG. 11B shows the sulfuric acid inlet 1108 into the manure/compost housing or pile 1110 results in the sulfuric acid-leached product provided out the outlet 1114, and where gas is released out the gas outlet 1106.

FIG. 11C shows the nitric/sulfuric acid inlet 1110 into the manure/compost housing or pile 1110 results in the nitric/sulfuric acid-leached product provided out the outlet 1116, and where gas is released out the gas outlet 1106.

As shown in FIGS. 11A, 11B, and 11C, the housing can be a tank, stirred tank, reactor, bioreactor, or the like. However, the manure/compost may not be in a housing, but may be piled or spread or in a basin or in a reservoir, or the housing may have an open top. In any event, the leached product can be obtained where the leaching may be in a housing or in another location. A leach pad, tank, or equivalent may also be used for leaching. The leaching with the acids may also result in the leached product having micronutrients, such as any micronutrients including those described herein or known in the art, such as the fertilizer art. The acid can be MPSA, MPNA, or any other microbially produced acid.

Additionally, any of the reagent sources, such as manure or compost, can be leached with water before, during or after any process or step as described herein. The water can leach minerals or ions from the reagent source, and such minerals and ions can be used to prepare the mineral salts or micro-nutrients described herein. In one example, many forms of phosphorus (P) and potassium (K) are soluble. As such, the water leaching can obtain phosphorus and potassium as part of the leachate, and the leachate can be used to make the mineral salts. That is, any bioreactor that prepares the mineral salts can include a leachate inlet, which leachate includes phosphorus, potassium or other substances for making the mineral salts. The leaching is shown in FIG. 11D. Also, the water leaching can occur in the silo.

In one embodiment, the leaching of FIGS. 11D can be applied to the compost silo of FIG. 5. In one example, water is introduced via a water inlet 112 into the silo (e.g., manure/compost housing or pile 1100) as a leaching liquid, which can cool the pile and leach out minerals and ions that are provided out the water-leached outlet 1118 and gas is removed from the gas outlet 1106. The leachate containing the minerals and ions can be collected at the bottom of the silo. The leachate can then be provided to a bioreactor.

In one aspect, the leached product can be introduced into any of the bioreactors, such as the bioreactors for preparing mineral salts. The leached product can be combined with products of processes performed in the bioreactors in order to make mineral salts. However, any of the mineral salts described herein or otherwise generally known can be prepared with the leached product.

In one aspect, the processes described herein can be performed with a feed gas from any source of dihydrogen sulfide. For example, any process, such as an industrial process, that emits dihydrogen sulfide can be used for a source of feed gas into a bioreactor that has microbes that process sulfur. The source for dihydrogen sulfide may also be from biological origins. Additionally, the source for dihydrogen sulfide may be from hydrocarbon sources. Some examples can include anaerobic digestion, sour oil and gas providing the dihydrogen sulfide.

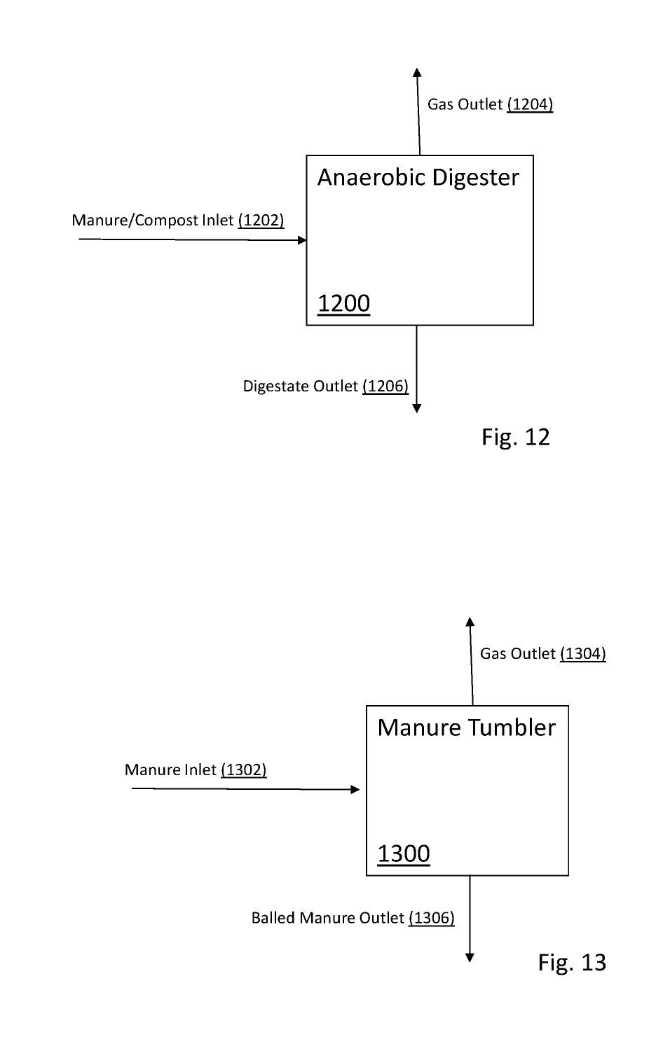

FIG. 12 shows an anaerobic digester system 1200 that can receive animal waste (e.g., manure/compost) via a manure/compost inlet 1202 and that has anaerobic microbes therein. The anaerobic microbes can digest the animal waste into a digestate, which is provided as a digestate outlet 1206. The digestate can be solid or liquid or combinations thereof, and may include gelatinous sludge or any form other than gas. The digestate can include any mineral salts or ions contained in the animal waste, such as potassium and phosphates. In one aspect, the digestate can be used as a reagent in any of the bioreactors. For example, the digestate may include potassium, and when combined with a bioreactor that makes sulfuric acid or sulfate mineral salt thereof, the potassium can facilitate the production of potassium sulfate (K2SO4). Also, phosphates in the digestate can facilitate the production of phosphoric acid (H3PO4). That is, the digestate can be provided as a digestate inlet to the bioreactors. The anaerobic digestion of the animal waste with the microbes can generate gases, which can be obtained in the gas outlet 1204. The gas outlet 1204 can be provided as a gas inlet (e.g., other gas inlet) to any of the bioreactors as shown. The gases can include methane (CH4), dihydrogen sulfide (H2S), carbon dioxide (CO2), and ammonia (NH3), or other gases. The digestate outlet can include a filter to separate solids (or any form other than liquids) from the liquids, whereby the solids and liquids of the digestate can be separated by filtration. The dihydrogen sulfide can be introduced into a bioreactor having microbes that produce MPSA and used in such process. In one aspect, the gas can be passed through a bioreactor to be scrubbed to result in a cleaner and/or better smelling gas (e.g., removal of dihydrogen sulfide).

In one aspect, the anaerobic digester system 1200 can include methanogens, which are microbes that produce methane, which can be considered to be archaea. The methanogens produce methane from carbon dioxide and hydrogen, or from acetic acid and hydrogen.

In one aspect, the anaerobic digester can include microbes that liberate hydrogen from the animal waste, which hydrogen may be used for protonation of phosphates. The microbes can produce biohydrogen, which is hydrogen produced biologically. The microbes that produce hydrogen can include algae (e.g., Chlamydomonas), bacteria (e.g., Clostidium, Escherichia, and Enterobacter), and some archaea, or others. The microbes may also use light to liberate hydrogen from animal waste.

FIG. 13 illustrates a manure tumbler 1300 can be used to prepare the manure for use in the systems and methods described herein. The manure tumbler 1300 can receive waste, such as manure and compost, at the manure inlet 1302. The manure tumbler 1300 can then tumble the manure until the waste balls up into spherical shapes. The manure tumbler 1300 can be configured with a drum that rotates about a horizontal axis that that the manure is tumbled. The manure tumbler 1300 can have air inlets to provide air flow into the manure to facilitate tumbling. Also, the manure tumbler 1300 may include a heating element to heat the manure as it tumbles to dry it out further. The gas outlet 1304 can release gases from the manure tumbler, and may provide the gases into any of the bioreactors herein. Often, the gases are those common with animal manure. The balled manure outlet 1306 can provide the balled waste to the bioreactors described herein, anaerobic digester, any manure/compost housing or pile, or to the composting silo. It has been found that the balled manure can provide enhanced operation and avoid plugging of the equipment. Also, the balled manure provides for interstitial spaces between the balled manure that allows for liquid and gas flow therebetween. Also, the balled manure can function as ball bearings and roll when being stiffed, such as in the compost silo. Thus, the processes described herein can be benefited by using balled manure.

In view of the foregoing descriptions, the present technology includes: microbial production of H2SO4 and HNO3; the use of these acids to produce other microbial acids such as phosphoric acid (e.g., with phosphate rock) and hydrochloric acid (e.g. with a source of chloride) as an intermittent step towards production of some microbial mineral salts (e.g., including phosphate mineral salts produced using microbes); the direct production of mineral salts from the acids, including but not limited to, ammonium sulfate (e.g., which can be mascagnite) and ammonium nitrate via injection of ammonia/ammonium, carbon dioxide, dihydrogen sulfide, hydrogen, methane, ammonia, combinations thereof, or any other mineral salt in accordance with the teachings provided herein from animal waste (e.g., gas from animal waste) whether being generated, stored, or composted; a method of composting manure to generate such gases quickly and efficiently using a stirred composting silo; and the capture of additional mineral substances from compost/manure via the use of MPSA and/or MPNA to leach the minerals out of the compost/manure and the subsequent conversion of these minerals into mineral salts or fertilizers.

In addition to the foregoing, the processes and bioreactors can be modified to produce any microbially produced acid (MPA; or biologically produced acid BPA). That is, any microbe that produces an acid can be introduced into the bioreactor along with the substrate the microbe processes to produce the MPA. Accordingly, the microbes may be capable of producing phosphoric acid, hydrochloric acid, and carbonic acids. Such microbes can be cultivated and cultured and selected to produce any acid, such as phosphoric acid, hydrochloric acid, and carbonic acids, or any other MPA. The MPAs produced as described herein can then be used to produce mineral salts, and thereby are microbially produced mineral salts (MPMS). The MPMS can be any salt of the MPAs having any suitable counter ion. This can include any sulfate salt, nitrate salt, phosphate salt, chloride salt, or carbonic salt (e.g., carbonate salt or bicarbonate salt), or any other. The counter ions can range from elements (e.g., potassium) to compounds (e.g., ammonium). Any known counter ion to the make these salts or any other MPMS can be used.

In one example, phosphoric acid can be a MPA.

In one example, hydrochloric acid can be a MPA that is formed by introducing KCl to a reactor as described herein or a pile with animal waste along with sulfuric acid (e.g., MPSA) to produce K2SO4 and HCl.

In one example, calcium can be a counter ion. The calcium can be introduced as a reagent in any way or form, such as in animal waste. As such, the mineral salts can be calcium sulfate or calcium nitrate.

In one example, carbonate salts can be produced as the mineral salts described herein. The counter ion of the carbonate salt can be any counter ion.

It should be recognized that any process used in the production of an acid, such as any MPA as described herein, can also simultaneously produce the mineral salt. In part, this is because the counter ion for a MPA to produce a mineral salt can be included in the reagents (e.g., manure, compost, etc.) that are in the bioreactor. Thus, any description of producing an acid with microbes also includes producing a mineral salt with a counter ion present with the acid.

In one embodiment, it should be noted that with other bioreactors, the microbial waste can be withdrawn as an outlet (e.g., microbial waste outlet). In some instance, such microbial waste may be a desired MPA.

It should be understood that the reagents in the processes described herein can be supplied as single components, such as pure ammonia or carbon dioxide, or multiple components, such as animal waste having ammonia and carbon dioxide. The reagents can be supplied as single atoms, molecules, or any combinations thereof.

Additionally, the ammonia may be considered to be from a source that is not organic. That is, the ammonia source is not certified as organic. The ammonia source can be from a Haber Bosch process or bottled gas or any synthetic or industrial process.

In view of the teachings herein and the skill of one of ordinary skill in the art, the bioreactors, such as shown in FIG. 4B, may be used for preparing a wide variety of minerals. The minerals may include sulfates and/or ammonium or other minerals that include sulfur and/or nitrogen. The minerals may not include sulfur and/or nitrogen. The mineral acid produced in the bioreactor may facilitate production of the mineral by the acid being incorporated into the mineral or by facilitating the complexing of acids and bases to form the mineral salts. The minerals may include water and may be hydrated, hydrates, or anhydrates that lack water included therein. The water used in the bioreactor may be regular water, pond water, river water, swamp water, processed water, tap water, city water, deionized water, electrolysis water, or any other water. As such, the water may include substances that may contribute to the formation of the mineral and that may be included in the minerals.

In one embodiment, the minerals may be any mineral that contains nitrogen. The nitrogen containing minerals may also include Al, C, H, Mg, Mn, SO4, PO4, K, Fe, H, Si, F, Cl, or other components. A list of minerals that can be prepared is provided in the table below. Generally, the minerals may be prepared in the bioreactor in accordance with the protocols described herein. In some instances, elements or substances having the elements of a mineral can be introduced into the bioreactor, such as through the reagent inlet. Any reagent that can be introduced into the bioreactor to produce the desired mineral may be used, which can include acids, bases, or salts or other minerals that include a desired element or substance to be included in the mineral. The microbially produced acids described herein may facilitate production of the minerals, and thereby the select microbes may be used for production of such minerals by producing the acids as part of the process for forming the minerals. The reagents may also be introduced in a stoichiometric or excess amount in order to guide formation of the desired mineral.

Nitrogen Mineral Table

| | |
|---|---|
| Abelsonite | Ni(C₃₁H₃₂N₄) |
| Acetamide | CH₃CONH₂ |
| Acmonidesite | (NH₄,K,Pb)₈NaFe₄²⁺(SO₄)₅Cl₈ |
| Adranosite-(Al) | (NH₄)₄NaAl₂(SO₄)₄Cl(OH)₂ |
| Adranosite-(Fe) | (NH₄)₄NaFe₂³⁺(SO₄)₄Cl(OH)₂ |
| Alumino- | (NH₄)₃Al(SO₄)₃ |
| pyracmonite | |
| Ambrinoite | [K,(NH₄)]₂(As,Sb)₆(Sb,As)₂S₁₃•H₂O |
| Ammineite | [CuCl₂(NH₃)₂] |
| Ammonioalunite | (NH₄)Al₃(SO₄)₂(OH)₆ |
| Ammonioborite | (NH₄)₂[B₅O₆(OH)₄]₂•H₂O |
| Ammoniojarosite | (NH₄)Fe₃³⁺(SO₄)₂(OH)₆ |
| Ammonioleucite | (NH₄,K)(AlSi₂O₆) |
| Ammonio- | (NH₄)₂Mg₅²⁺Fe₃³⁺Al(SO₄)₁₂•18H₂O |
| magnesiovoltaite | |
| Archerite | (K,NH₄)(H₂PO₄) |
| Argesite | (NH₄)₇Bi₃Cl₁₆ |
| Bararite | (NH₄)₂[SiF₆] |
| Barberiite | (NH₄)[BF₄] |
| Beshtauite | (NH₄)₂(UO₂)(SO₄)₂•2H₂O |
| Biphosphammite | (NH₄,K)(H₂PO₄) |
| Boussingaultite | (NH₄)₂Mg(SO₄)₂•6H₂O |
| Brontesite | (NH₄)₃PbCl₅ |
| Buddingtonite | (NH₄)(AlSi₃)O₈ |
| Buttgenbachite | Cu₁₉(NO₃)₂(OH)₃₂Cl₄•2H₂O |
| Carlsonite | (NH₄)Fe₃³⁺O(SO₄)₆•7H₂O |
| Chanabayaite | CuCl(N₃C₂H₂)(NH₃)•0.25H₂O |
| Clairite | (NH₄)₂Fe₃(SO₄)₄(OH)₃•3H₂O |
| Comancheite | Hg₅₅²⁺N₂₄³⁻(NH₂,OH)₄(Cl,Br)₃₄ |
| Cryptohalite | (NH₄)₂[SiF₆] |
| Darapskite | Na₃(SO₄)(NO₃)•H₂O |
| Dittmarite | (NH₄)Mg(PO₄)•H₂O |
| Efremovite | (NH₄)₂Mg₂(SO₄)₃ |
| Gerhardtite | Cu₂(NO₃)(OH)₃ |
| Godovikovite | (NH₄)Al(SO₄)₂ |
| Guanine | C₅H₅N₅O |
| Gwihabaite | (NH₄,K)NO₃ |
| Hannayite | (NH₄)₂Mg₃H₄(PO₄)₄•8H₂O |
| Huizingite-(Al) | [(NH₄)₉(SO₄)₂][(Al,Fe³⁺)₃(OH)₂(H₂O)₄(SO₄)₆] |
| Humberstonite | Na₇K₃Mg₂(SO₄)₆(NO₃)₂•6H₂O |
| Hydrombobomkulite | (Ni,Cu)Al₄((NO₃)₂,SO₄)(OH)₁₂•13-14H₂O |
| Joanneumite | Cu(C₃N₃O₃H₂)₂(NH₃)₂ |
| Julienite | Na₂[Co(SCN)₄]•8H₂O |
| Kalinite | KAl(SO₄)₂•11H₂O |
| Kladnoite | C₆H₄(CO)₂NH |
| Kleinite | (Hg₂N)(Cl,SO₄)•nH₂O |
| Koktaite | (NH₄)₂Ca(SO₄)₂•H₂O |
| Kremersite | (NH₄,K)₂[Fe³⁺Cl₅(H₂O)] |
| Larderellite | (NH₄)B₅O₇(OH)₂•H₂O |
| Lecontite | (NH₄,K)NaSO₄•2H₂O |
| Letovicite | (NH₄)₃H(SO₄)₂ |
| Likasite | Cu₃(NO₃)(OH)₅•2H₂O |
| Lislkirchnerite | Pb₆Al(OH)₈Cl₂(NO₃)₅•2H₂O |
| Lonecreekite | (NH₄)Fe³⁺(SO₄)₂•12H₂O |
| Lucabindiite | (K,NH₄)As₄O₆(Cl,Br) |
| Mascagnite | (NH₄)₂SO₄ |
| Mbobomkulite | (Ni,Cu)Al₄((NO₃)₂,SO₄)(OH)₁₂•3H₂O |
| Melanophlogite | 46SiO₂•6(N₂,CO₂)•2(CH₄,N₂) |
| Möhnite | (NH₄)K₂Na(SO₄)₂ |
| Mohrite | (NH₄)₂Fe(SO₄)₂•6H₂O |
| Mosesite | (Hg₂N)(Cl,SO₄,MoO₄)•H₂O |
| Mundrabillaite | (NH₄)₂Ca(HPO₄)₂•H₂O |
| Niahite | (NH₄)(Mn²⁺,Mg)(PO₄)•H₂O |
| Nickel- | (NH₄)₂Ni(SO₄)₂•6H₂O |
| boussingaultite | |
| Nierite | Si₃N₄ |
| Niter | KNO₃ |
| Nitratine | NaNO₃ |
| Nitrocalcite | Ca(NO₃)₂•4H₂O |
| Nitromagnesite | Mg(NO₃)₂•6H₂O |
| Oxammite | (NH₄)₂(C₂O₄)•H₂O |
| Panichiite | (NH₄)₂SnCl₆ |
| Phosphammite | (NH₄)₂(HPO₄) |
| Pyracmonite | (NH₄)₃Fe(SO₄)₃ |
| Roaldite | (Fe,Ni)₄N |
| Rouaite | Cu₂(NO₃)(OH)₃ |
| Sabieite | (NH₄)Fe³⁺(SO₄)₃ |
| Salammoniac | NH₄Cl |
| Schertelite | (NH₄)₂MgH₂(PO₄)₂•4H₂O |
| Schindlerite | {(NH₄)₄Na₂(H₂O)₁₀}{V₁₀O₂₈} |
| Shilovite | Cu(NH₃)₄(NO₃)₂ |
| Sinoite | Si₂N₂O |
| Spheniscidite | (NH₄,K)(Fe³⁺,Al)₂(PO₄)₂(OH)•2H₂O |
| Stercorite | Na(NH₄)HPO₄•4H₂O |
| Struvite | (NH₄)Mg(PO₄)•6H₂O |
| Suhailite | (NH₄)Fe₃²⁺(AlSi₃O₁₀)(OH)₂ |
| Sveite | KAl₇(NO₃)₄(OH)₁₆Cl₂•8H₂O |
| Swaknoite | (NH₄)₂Ca(HPO₄)₂•H₂O |
| Taranakite | (K,NH₄)Al₃(PO₄)₃(OH)•9H₂O |
| Teschemacherite | (NH₄)HCO₃ |
| Therasiaite | (NH₄)₃KNa₂Fe²⁺Fe³⁺(SO₄)₃Cl₅ |
| Thermessaite-(NH4) | (NH₄)₂(AlF₃)(SO₄) |
| Tobelite | (NH₄,K)Al₂(AlSi₃O₁₀)(OH)₂ |
| Tsaregorodtsevite | (N(CH₃)₄)(AlSi₅O₁₂) |
| Tschermigite | (NH₄)Al(SO₄)₂•12H₂O |
| Ungemachite | K₃Na₈Fe(SO₄)₆(NO₃)₂•6H₂O |
| Uramarsite | (NH₄,H₃O)₂(UO₂)₂(ASO₄,PO₄)₂•6H₂O |
| Uramphite | (NH₄)₂(UO₂)₂(PO₄)₂•6H₂O |
| Urea | CO(NH₂)₂ |
| Uricite | C₅H₄N₄O₃ |
| Wernerbaurite | {(NH₄)₂ [Ca₂(H₂O)₁₄](H₂O)₂}{V₁₀O₂₈} |
| Witzkeite | Na₄K₄Ca(NO₃)₂(SO₄)₄•2H₂O |

In one embodiment, the minerals may be any mineral that contains sulfate. The sulfate containing minerals may also include Al, C, H, Mg, Mn, SO4, PO4, K, Fe, H, Si, F, Cl, or other components. A list of minerals that can be prepared is provided in the table below. Generally, the minerals may be prepared in the bioreactor in accordance with the protocols described herein. In some instances, elements or substances having the elements of a mineral can be introduced into the bioreactor, such as through the reagent inlet. Any reagent that can be introduced into the bioreactor to produce the desired mineral may be used, which can include acids, bases, or salts or other minerals that include a desired element or substance to be included in the mineral. The microbially produced acids described herein may facilitate production of the minerals, and thereby the select microbes may be used for production of such minerals by producing the acids as part of the process for forming the minerals. The reagents may also be introduced in a stoichiometric or excess amount in order to guide formation of the desired mineral.

Sulfate Mineral Table

Barite (BaSO₄)
Celestite (SrSO₄)
Anglesite (PbSO₄)
Anhydrite (CaSO₄)
Hanksite (Na₂₂K(SO₄)₉(CO₃)₂Cl)
Gypsum (CaSO₄•2H₂O)
Chalcanthite (CuSO₄•5H₂O)
Kieserite (MgSO₄•H₂O)
Starkeyite (MgSO₄•₄H₂O)
Hexahydrite (MgSO₄•6H₂O)
Epsomite (MgSO₄•7H₂O)
Meridianiite (MgSO₄•11H₂O)
Melanterite (FeSO₄•7H₂O)
Antlerite (Cu₃SO₄(OH)₄)
Brochantite (Cu₄SO₄(OH)₆)
Alunite (KAl₃(SO₄)₂(OH)₆)
Jarosite (KFe₃(SO₄)₂(OH)₆)

The minerals that may be formed can have various uses. In some examples, the minerals may be biologically compatible and non-toxic. Such minerals may be used as fertilizers or have other agricultural or industrial uses.

In some instances, the inputs into the reactor may have a contaminant or undesirable element, and the minerals can be used to capture that contaminant or undesirable element. This allows the minerals having such undesirable elements to be prepared, collected, and then processed appropriately, which may include burying or storing such minerals. Some minerals may be avoided and not produced by selectively excluding one or more elements thereof or substances that have such elements that are not desirable. From the tables above, minerals that have the following elements or substances can either be prepared to entrap the elements or may be affirmatively not made by excluding the following elements or substances: Hg, U, B, Pb, Ni, Sb, As, Bi, Co, CN, Mo, Sn, V, or combinations thereof. As such, in one aspect, the minerals produced by the protocols may affirmatively exclude minerals that have these elements/substances, which can be accomplished by omitting such elements/substances from the bioreactor. In most cases, such elements/substances can be omitted by merely not introducing such elements/substances into the bioreactor. Also, the processes can include selectively removing such elements/substances from the feed or reagents to the bioreactor.

When a certain metal or element that may not be obtained from compost, or manure, or other animal waste, such metal or element may be affirmatively introduced into the bioreactor. In some instances, the feeds to the bioreactor may be deficient or lack a suitable amount of such metal or element, and thereby the protocols for forming minerals that include such a metal or element may include affirmatively introducing the metal or element or source for such metal or element into the bioreactor. Such metals or elements may include: Ca, Fe, Ni, Al, C, H, Mn, SO4, PO4, K, H2, Sb, As, Cu, Mg, Si, F, Cl, Co, CN, Mo, Sn, V, CH3, or combinations thereof. In one aspect, such metals or elements may include: Ca, Fe, Al, C, H, Mn, SO4, PO4, K, H2, Cu, Mg, Si, F, Cl, CH3, or combinations thereof. In one aspect, such metals or elements may include: Ca, Fe, Al, C, H, Mn, SO4, PO4, K, H2, Mg, or combinations thereof. In some instances the feed or reagents to the bioreactor can include these elements/substances, or such elements and substances can be added to the feed or reagents or introduced directly to the bioreactor. Thus, in one aspect, the minerals produced by the protocols may affirmatively include minerals that have these elements.

The bioreactor can be operated as described herein with distilled water in order to obtain the recited minerals in the table. The feed and reagents introduced into the bioreactor can be selected and proportioned in order to prepare the desired mineral, such as from the table. Experiments may also be conducted at certain feed and reagent amounts or ratios in order to determine the amount of element/substance to include in the bioreactor with the rest of the feed and reagents under the operating conditions. If too much or too little of a certain element/substance does not result in the desired mineral, the amounts can be adjusted and iterative experiments and analyses can be conducted until it is determined that the desired mineral is being produced.

In one example, it was determined that koktaite can be produced in the bioreactor operated as described herein when Ca is also included in the bioreactor. The Ca can react with the other components in order to form the koktaite mineral. During the process, the bioreactor formed about 65% mascagnite, 25% koktaite with the balance being a mixture of gypsum and anhydrite. It is thought that varying the amount of available Ca can vary the amount of koktaite that can be produced. The produced mineral mixture can then be purified to obtain purified mineral without the other minerals.

In one embodiment, the recited elements/substances may be included in a feed or reagent to a bioreactor by leaching animal waste or compost with an acid, such as a microbial acid described herein. The leachate may also include micronutrients that may be incorporated into the mineral salts.

In one embodiment, a system can include a plurality of bioreactors. In such a system, the bioreactors can be selected to receive certain feeds or reagents in order to produce selected mineral salt(s) in one reactor and selected mineral salt(s) in other reactors. The off gas or vented gas from one bioreactor may be recycled to the same reactor or different reactor in the system. Similarly, the liquid medium of one bioreactor can be withdrawn and recycled back to the same reactor or to a different reactor in the system. Such selection and use of multiple bioreactors in a system can be tailored to optimize mineral production based on the chemical component distributions of the different feeds and reagents into the bioreactors, the resulting products from the bioreactors, and selection and use thereof. The different minerals may also be precipitated in sequences based on the contents of the bioreactor media.

In one example, a bioreactor system can be operated so as to produce a first mineral in a first reactor (e.g., mascagnite), and operated so as to produce a second mineral in a second reactor (e.g., nitrogen containing mineral) and operated so as to produce a third mineral in a third reactor (e.g., sulfate containing mineral). The first mineral, second mineral or third mineral may optionally be a combination of mineral salts. The reactors may be fed with compost leachate, H2SO3, nitric acid, phosphoric acid other acid, or compost/manure off gas. Alternatively, the acids may be generated by microbes in the reactors. The process can include precipitating different salts sequentially out of solution and collecting the different salts separately or sequentially, or collecting a mixture or mineral salts. The process can include manipulating the reactor conditions and feed inputs, controlling feed inputs and reactor conditions to produce a desired mineral salt, such as a sulfate mineral salt or a nitrogen containing mineral salt.

In one embodiment, a method of producing sulfuric acid can include: obtaining a microbial culture that produces sulfuric acid; placing the microbial culture in a bioreactor in an aqueous environment; introducing an aqueous, liquid or gaseous sulfur supply into the bioreactor; and culturing the microbial culture with the sulfur supply sufficiently so that sulfuric acid is produced. In one aspect, the sulfur supply is from gaseous sulfur dioxide and/or dihydrogen sulfide. In one aspect, the sulfur supply is aqueous sulfur dioxide and/or dihydrogen sulfide. In one aspect, the sulfur supply is sulfurous acid. In one aspect, the produced sulfuric acid is MPSA from an organic sulfur supply. In one aspect, the microbes are any microbes that processes sulfur. In one aspect, the microbes are any natural microbes that processes sulfur. In one aspect, the microbes are any genetically modified microbes that processes sulfur. In one aspect, the microbes are any cultivated microbes that processes sulfur. In one aspect, the microbes are any purchased microbes that processes sulfur. In one aspect, the microbes include other type of microbes that facilitate culturing of the sulfuric acid producing microbes. In one aspect, the sulfuric acid is produced at ambient conditions. In one aspect, the process is a batch process. In one aspect, the process is a continuous process.

In one embodiment, the method can include: burning sulfur to obtain gaseous sulfur dioxide; and bubbling gaseous sulfur dioxide through the aqueous environment of the bioreactor having the microbes. In one aspect, the method can include: burning sulfur to obtain gaseous sulfur dioxide; mixing the sulfur dioxide with water to form aqueous sulfur dioxide; and injecting aqueous sulfur dioxide into the aqueous environment of the bioreactor having the microbes. In one aspect, the method can include: burning sulfur to obtain gaseous sulfur dioxide; mixing the sulfur dioxide with water to form sulfurous acid; and injecting sulfurous acid into the aqueous environment of the bioreactor having the microbes. In one aspect, the bioreactor is part of a sulfurous acid producing system.

In one aspect, the bioreactor receives sulfurous acid from a sulfurous acid producing system.

In one embodiment, the microbes freely float or are suspended in the aqueous environment. In one aspect, the microbes are on a substrate that freely float or are suspended in the aqueous environment. In one aspect, the microbes are on a substrate affixed to a surface of the bioreactor.

In one embodiment, the method can include: recycling an aqueous liquid from the bioreactor through a sulfurous acid producing system until reaching a threshold pH, the aqueous liquid having a higher pH than the threshold; and once the threshold pH is reached, extracting sulfuric acid from the bioreactor. In one aspect, the method can include: measuring the pH of the aqueous environment; and electively extracting sulfuric acid when the pH is lower than the threshold pH; and selectively recycling sulfurous acid when the pH is above the threshold pH. In one aspect, the method can include producing sulfuric acid until at least 0.1%, 1% 10%, 29%, 32%, 62%, 70%, 78%, 80%, or 98% or any range between any of the values of sulfuric acid.

In one embodiment, the method can include: anaerobically digesting animal waste to produce digestate; and providing the digestate to the bioreactor. In one aspect, the method can include: anaerobically digesting animal waste to produce digestate gas; and providing the digestate gas to the bioreactor.

In one embodiment, a method can include culturing microbes that produce sulfuric acid by: locating a geothermal area; obtaining a microbial culture from the geothermal area; selecting microbes from the microbial culture that produce sulfuric acid; and culturing the selected microbes with an aqueous, liquid or gaseous sulfur supply. In one aspect, the geothermal area includes a fumarole, hot pot, warm spring, geyser, hot vent, volcanic formation, or any other geological formation. In one aspect, the method can include culturing microbes that produce sulfuric acid by: locating geologic area that is contaminated and has acidic water; obtaining a microbial culture from the contaminated geologic area; selecting microbes from the microbial culture that produce sulfuric acid; and culturing the selected microbes with an aqueous, liquid or gaseous sulfur supply. In one aspect, the contaminated geologic area includes a mine having mine waste water or mine tailings or mineral processing plant or waste stream or tailings or dump or anywhere there is a sulfur contamination.

In one embodiment, a method of producing ammonium sulfate can include: obtaining a microbial culture that produces sulfuric acid; placing the microbial culture in a bioreactor in an aqueous environment; introducing a solid, particulate, aqueous, liquid or gaseous sulfur supply into the bioreactor; introducing an ammonia source into the bioreactor and culturing the microbial culture with the sulfur supply and ammonia source sufficiently so that sulfuric acid is produced that reacts with the ammonia so as to produce ammonium sulfate. In one aspect, the sulfuric acid produced by microbes as described herein can be used to produce the ammonium sulfate. In one aspect, the sulfur supply is from gaseous sulfur dioxide. In one aspect, the sulfur supply is from solid sulfur. In one aspect, the sulfur supply is from particulate sulfur. In one aspect, the sulfur supply is from particulate sulfur having a dimension less than 37 microns. In one aspect, the sulfur supply is from particulate sulfur having a dimension less than 1 micron. In one aspect, the sulfur supply is from a sulfur mineral. In one aspect, the sulfur supply is from a sulfide. In one aspect, the sulfur supply is from pyrite. In one aspect, the sulfur supply is aqueous sulfur dioxide. In one aspect, the sulfur supply is sulfurous acid. In one aspect, the sulfur is solid sulfur used to prepare the ammonium sulfate. In one aspect, the microbes are any microbes that processes sulfur, such as those described herein. In one aspect, the microbes include other type of microbes that facilitate culturing of the sulfuric acid producing microbes. The sulfur burning system and sulfur burning methods may also be used to provide gaseous sulfur dioxide to water in the bioreactor, which can provide sulfurous acid as described herein, which can be used to prepare the ammonia sulfate. In one embodiment, the sulfuric acid and ammonium sulfate are produced at ambient conditions in the bioreactor or sulfur burning system having the bioreactor. The microbes can be the same as those used to make sulfuric acid. The process can be batch or continuous. Liquid recycling can also be used to obtain the sulfuric acid as a desired pH as described herein. The method can also include culturing the sulfuric acid producing microbes and obtaining such microbes from the environment or selecting such microbes.

In one embodiment, the MPSA used to make the ammonia sulfate is biologic sulfuric acid from an allowed organic sulfur supply, the ammonia is from an organic ammonia supply, and the resulting ammonium sulfate is therefore organic. In one aspect, the produced sulfuric acid is MPSA from an organic sulfur supply, the salt-producing reagent is from an organic supply, and the resulting mineral salt is organic.

In one embodiment, the ammonia source is organic. In one aspect, the ammonia source is from excrement, compost, feces, urine or combination thereof. In one aspect, the ammonia source is from a Berkley Technique composting silo. In one aspect, the process can make the smell of a farm better by making ammonium sulfate from animal waste. Digestate may be made and used in the process.

In one embodiment, the method can include collecting the ammonium sulfate as a precipitate. In one aspect, the method can include: withdrawing liquid from the bioreactor; and processing the liquid to obtain the ammonium sulfate. In one aspect, the method can include: withdrawing liquid from the bioreactor; and centrifuging the liquid to obtain the ammonium sulfate. In one aspect, the method can include recycling liquid from the centrifuge back to the bioreactor. In one aspect, the process can use equipment to remove water from the ammonium sulfate. In one aspect, the method can include using equipment to remove ammonium sulfate from the aqueous environment.

In one embodiment, the method can include: anaerobically digesting animal waste to produce digestate; and providing the digestate to the bioreactor.

In one embodiment, the method can include: introducing a phosphate to the MPSA; and producing a phosphate mineral salt. In one aspect, the method ca include: introducing a rock or milled particle thereof having phosphate or derivative there to the MPSA; and producing a phosphate mineral salt. In one aspect, the method can include: introducing a phosphate to the MPSA to produce phosphoric acid; and producing a phosphate mineral salt from the phosphoric acid and counter ions from reagents in the bioreactor. In one aspect, the method can include: introducing a phosphate to the MPSA to produce phosphoric acid.

In one embodiment, the method can include: introducing a carbonic acid or carbonate to the MPSA; and producing a carbonate mineral salt.

In one embodiment, a Berkley Technique for a composting silo can be used to prepare reagents for preparing the microbially produced acids and minerals as described herein. As such, the composting method can include using a silo comprising: a silo housing defining a silo cavity; insulation on a surface of the silo housing around the silo cavity; one or more mixing devices; an air inlet at the bottom of the silo cavity; an ammonia air outlet at the top of the silo cavity; and a compost outlet at the bottom of the silo cavity. In one aspect, the ammonia air outlet is fluidly coupled to a bioreactor that makes ammonium salt with microbes, such as ammonium nitrate and/or ammonium sulfate and/or ammonium phosphate. In one aspect, the mixing devices are augers that extend longitudinally in the silo cavity. In one aspect, the mixing devices are arms that can move within the silo cavity. In one aspect, the silo is used in a composting process that modulates pH during composting. In one aspect, the silo is used in a composting process that increases the pH. In one aspect, the silo is used in a composting process that introduces an alkaline material into the silo cavity. In one aspect, the silo is used in a composting process that modulates the carbon : nitrogen ratio during composting. In one aspect, the silo is used in a composting process that reduces the carbon:nitrogen ratio. In one aspect, the silo is used in a composting process that introduces microbes to the compost in the silo cavity. In one aspect, the silo can include a microbe reservoir having EM-1 microbes.

In one embodiment, a composting method can include: providing a silo described herein; introducing animal waste into the silo; and compositing the animal waste in order to generate gasses from the animal waste. In one aspect, the method can include collecting the generated gases from the silo. In one aspect, the generated gases include ammonia and/or ammonium. In one aspect, the method can include mixing the animal waste with augurs during the compositing. In one aspect, the method can include passing air through the animal waste during the compositing. In one aspect, the method can include collecting compost from the silo cavity. In one aspect, the method can include modulating the pH of the animal waste during the composting. In one aspect, the method can include introducing microbes during the compositing. In one aspect, the microbes include EM-1 microbes.

In one embodiment, any method described herein can include: leaching the animal waste or compost therefrom with water; and collecting the leachate, which can be used in any of the bioreactors for any of the processes. In one aspect, the leachate has potassium and/or phosphorous or salts thereof. In one aspect, the method can include: leaching the animal waste or compost therefrom with acid; and collecting the leachate. In one aspect, the leachate has potassium and/or phosphorous or salts thereof. In one aspect, the acid used for leaching is a microbially produced acid, such as described herein. In one aspect, the method can include: anaerobically digesting animal waste to produce digestate; and providing the digestate to the silo. In one aspect, the method can include introducing reagents into the silo that facilitate composting of the animal waste before, during or after introducing the animal waste into the silo. In one aspect, the method can include composting as described herein with any silo.

In one embodiment, a method of producing nitrite can include: obtaining a microbial culture that produces nitrite; placing the microbial culture in a bioreactor in an aqueous environment; introducing an aqueous, liquid or gaseous ammonia and/or ammonium supply into the bioreactor; and culturing the microbial culture with the ammonia and/or ammonium supply sufficiently so that nitrite is produced. In one aspect, the ammonia or ammonium supply is from animal waste. In one aspect, ammonia and/or ammonium is used. In one aspect, the produced nitrite is organic nitrite from an organic nitrogen supply.

In one embodiment, the microbes are any microbes that processes nitrogen-containing compounds into nitrite. In one aspect, the microbes are any natural microbes that processes nitrogen. In one aspect, the microbes are any genetically modified microbes that processes nitrogen. In one aspect, the microbes are any cultivated microbes that processes nitrogen. In one aspect, the microbes are any purchased microbes that processes nitrogen. In one aspect, the microbes include other types of microbes that facilitate culturing of the nitrate or nitric acid producing microbes.

In one embodiment, the ammonia is from any source, such as a natural source, a synthetic or industrial source, an organic ammonia source, or not from an organic ammonia source.

In one embodiment, a method of producing nitrite can include: obtaining a microbial culture that produces nitrate; placing the microbial culture in a bioreactor in an aqueous environment; introducing an aqueous, liquid or gaseous ammonia and/or ammonium supply into the bioreactor; and culturing the microbial culture with the ammonia and/or ammonium supply sufficiently so that nitrate is produced. Any ammonia or ammonium supply can be used, such as from animal waste or compost, or the like. In one aspect, the method can include forming nitrite with microbes that is then processed into nitrate microbes. In one aspect, the produced nitrate is organic nitrite from an organic nitrogen supply. In one aspect, the microbes are any microbes that processes nitrogen-containing compounds into nitrate. In one aspect, the microbes are any natural microbes that processes nitrogen. In one aspect, the microbes are any genetically modified microbes that processes nitrogen. In one aspect, the microbes are any cultivated microbes that processes nitrogen. In one aspect, the microbes are any purchased microbes that processes nitrogen. In one aspect, the microbes include other types of microbes that facilitate culturing of the nitrate or nitric acid producing microbes. In one aspect, the ammonia is from any source, such as described herein.

In one embodiment, a method of producing nitric acid can include: obtaining a microbial culture that produces nitrite or nitrate; placing the microbial culture in a bioreactor in an aqueous environment; introducing an aqueous, liquid or gaseous ammonia and/or ammonium supply into the bioreactor; culturing the microbial culture with the ammonia and/or ammonium supply sufficiently so that nitrite or nitrate is produced; and processing the nitrite or nitrate into nitric acid. In one aspect, the ammonia or ammonium supply is from animal waste or any other source. In one aspect, the produced nitrite or nitrate is organic nitrite or nitrate from an organic nitrogen supply. In one aspect, the microbes are any microbes that processes nitrogen-containing compounds into nitrite or nitrate, such as those described herein. In one aspect, the microbes include other type of microbes that facilitate culturing of the nitrite or nitrate producing microbes.

In one embodiment, the method can include: introducing a phosphate to the nitric acid; and producing a phosphate mineral salt. In one aspect, the method can include: introducing a rock or milled particle thereof having phosphate or derivative there to the nitric acid; and producing a phosphate mineral salt. In one aspect, the method can include: introducing a phosphate to the nitric acid to produce phosphoric acid; and producing a phosphate mineral salt from the phosphoric acid and counter ions from reagents in the bioreactor. In one aspect, the method can include: introducing a phosphate to the nitric acid to produce phosphoric acid.

In one embodiment, the method can include: introducing a carbonic acid or carbonate to the nitric acid; and producing a carbonate mineral salt.

In one embodiment, a method of producing a sulfate mineral salt can include: obtaining a microbial culture that produces sulfuric acid; placing the microbial culture in a bioreactor in an aqueous environment; introducing an aqueous, liquid or gaseous sulfur supply into the bioreactor; introducing a mineral salt-producing reagent source into the bioreactor and culturing the microbial culture with the sulfur supply and mineral salt-producing reagent source sufficiently so that sulfuric acid is produced that reacts with the mineral salt-producing reagent so as to produce the sulfate mineral salt. In one aspect, the sulfur supply is from gaseous sulfur dioxide or sulfurous acid or any solid supply, such as described herein: solid sulfur; particulate sulfur; particulate sulfur having a dimension less than 37 microns; particulate sulfur having a dimension less than 1 micron; a sulfur mineral; a sulfide; pyrite; sulfur supply is aqueous sulfur dioxide. In one aspect, the produced sulfuric acid is MPSA from an organic sulfur supply, the mineral salt-producing reagent is from an organic supply, and the resulting fertilizer is organic. In one aspect, the microbes are any microbes (e.g., natural geneticially modified and the like) that process sulfur, such as described herein, and can optionally include other type of microbes that facilitate culturing of the sulfuric acid producing microbes. The microbes can be floating or on substrates as described herein. Also, the sulfur burning system and products thereof can be used to supply the sulfur, and wherein the bioreactor can be part of a sulfurous acid producing system. The system can be batch or continuous. The process can include recycling liquid, such as to obtain a desired pH. The method can include producing and using a digestate as described herein. Also, the process can include obtaining the proper microbes, selecting the microbes as needed, and culturing the microbes to be able to produce the mineral salts. The method can also include using phosphates to make phosphate minerals as described herein. The method can also include making carbonates as described herein. The sulfate mineral can be any of those listed herein that include a sulfate component, such as those in the tables.

In one embodiment, the mineral salt-producing reagent source is organic. For example, the mineral salt-producing reagent source is from excrement, compost, feces, urine or combination thereof. In one aspect, the mineral salt-producing reagent source is from a Berkley Technique composting silo.

The method can include collecting the mineral salt as a precipitate by any of the methods of collecting any of the mineral salts described herein.

In one embodiment, the mineral salt is Arcanite (K2SO4), Langbeinite, Anhydrite/Gypsum (CaSO4), and Epsomite (Epsom Salts) (MgSO4). In one aspect, the mineral salt is a sulfate, nitrate, and/or phosphate. In one aspect, the mineral salt is produced using MPSA. In one aspect, the mineral salt is any mineral salt that include phosphates, nitrates, and sulfates. In one aspect, the mineral salt is ammonium sulfate, potassium sulfate, potassium magnesium sulfate, gypsum (e.g., calcium sulfate), sulfur-containing nitrogen or nitrogen phosphorus potassium (NPK) salts. In one aspect, the mineral salt is produced with the MPSA and includes nitrogen, phosphorus, or potassium; nitrogen-sulfur, such as ammonium sulfate, ammonium nitrate-sulfate, ammonium phosphate-sulfate, and ammonium phosphate-nitrate; and potassium-sulfur mineral salts such as potassium sulfate and potassium magnesium-sulfate. In one aspect, the mineral salt includes micronutrients such as ZnSO4 and FeSO4, or others.

In one embodiment, the sulfate mineral can be formed to be devoid of one or more of the following elements or substances: Hg, U, B, Pb, Ni, Sb, As, Bi, Co, CN, Mo, Sn, V, or combinations thereof, or devoid of Hg, U, B, Pb, Sb, As, Bi, Mo, or combinations thereof. The sulfate mineral can be any of those listed devoid of these components.

In one embodiment, the sulfate mineral can be formed to include one or more of the following elements or substances: Ca, Fe, Ni, Al, C, H, Mn, SO4, PO4, K, H2, Sb, As, Cu, Mg, Si, F, Cl, Co, CN, Mo, Sn, V, CH3, or combinations thereof; or Ca, Fe, Al, C, H, Mn, SO4, PO4, K, H2, Cu, Mg, Si, F, Cl, CH3, or combinations thereof; or Ca, Fe, Al, C, H, Mn, SO4, PO4, K, H2, Mg, or combinations thereof. The sulfate mineral can be any of those listed with these components.

In one embodiment, a method of producing a sulfate and/or nitrate mineral salt can include: obtaining a microbial culture that produces sulfuric acid and/or nitric acid; placing the microbial culture in a bioreactor in an aqueous environment; introducing an aqueous, liquid or gaseous sulfur supply and/or nitrogen supply into the bioreactor; introducing a mineral salt-producing reagent source into the bioreactor; and culturing the microbial culture with the sulfur supply and/or nitrogen supply and mineral salt-producing reagent source sufficiently so that sulfuric acid and/or nitric acid is produced that reacts with the mineral salt-producing reagent so as to produce the sulfate and/or nitrate mineral salt. The sulfur supply can be from any sulfur substance as described herein. The nitrogen supply can be from any nitrogen substance as described herein. The microbes are any microbes that process sulfur and/or nitrogen. The microbes can include other type of microbes that facilitate culturing of the sulfuric acid producing microbes and/or nitric acid producing microbes. This method can include the use of the sulfur burning system and sulfur products thereof. The process can be batch or continuous. The process may use recycling to obtain a desired pH. The sulfate and/or nitrate mineral salt can be collected as with any mineral salt. The mineral salt can be any that includes sulfate and/or nitrate, and may optionally include a phosphate, and may include any micronutrient. The process can include making and using a digestate as a reagent. The process may also include using phosphate to include phosphates in the mineral salt. Also, carbonate mineral salts can be prepared by using carbonic acid or carbonate.

In one embodiment, the method can include producing nitrite to produce the nitric acid. In one aspect, the method can include producing nitrate to produce the nitric acid. In one aspect, the method can include producing nitrate instead of the nitric acid; and preparing the mineral salt from the nitrate.

In one embodiment, a method of producing a nitrate mineral salt can include: obtaining a microbial culture that produces nitrite and/or nitrate and/or nitric acid; placing the microbial culture in a bioreactor in an aqueous environment; introducing an aqueous, liquid or gaseous nitrogen supply into the bioreactor; introducing a mineral salt-producing reagent source into the bioreactor; and culturing the microbial culture with the nitrogen supply and mineral salt-producing reagent source sufficiently so that nitrite and/or nitrate and/or nitric acid is produced that reacts with the mineral salt-producing reagent so as to produce the nitrate mineral salt. The method can be performed with the steps for preparing other mineral salts described herein that result in nitrate minerals. As such, the nitrogen can be from any nitrogen supply. The microbes can be the appropriate microbes that process nitrogen substances as described herein. The methods of producing the nitrogen reagent, such as using the silo and/or manure, may be applied. In one aspect, the mineral salt is calcium nitrate, ammonium nitrate, calcium ammonium nitrate, sodium nitrate, potassium nitrate, magnesium nitrate, or combination thereof. The method can include producing nitrite to produce the nitric acid or nitrate to produce the nitric acid. Phosphates and carbonates may also be include and prepared as described herein.

In one embodiment, a method for producing a microbial acid can include: providing a microbe that converts a substrate to an acid; providing the substrate; and culturing the microbe sufficiently to produce the microbial acid.

In one embodiment, a method for producing a mineral salt can include: providing a microbe that converts a substrate to an acid; providing the substrate; culturing the microbe sufficiently to produce the microbial acid; introducing a counter ion for the microbial acid; and producing a mineral salt from the microbial acid and counter ion.

In one embodiment, a method for producing a microbial phosphoric acid can include: providing a microbe that converts a substrate to phosphoric acid; providing the substrate; and culturing the microbe sufficiently to produce the microbial acid.

In one embodiment, a method for producing a mineral phosphate salt can include: providing a microbe that converts a substrate to a phosphoric acid; providing the substrate; culturing the microbe sufficiently to produce the microbial phosphoric acid; introducing a counter ion for the microbial phosphoric acid; and producing a mineral salt from the microbial phosphoric acid and counter ion. These methods can be performed in bioreactors as described herein.

In one embodiment, a method of producing a nitrogen containing mineral salt can include: placing a microbial culture in a bioreactor in an aqueous environment, the microbial culture produces an acid; introducing one or more mineral salt-producing reagent sources having at least one nitrogen substance into the bioreactor; producing the acid; and culturing the microbial culture with the one or more mineral salt-producing reagent sources sufficiently so that the acid that is produced reacts with the mineral salt-producing reagent so as to produce the nitrogen containing mineral salt. In one aspect, the one or more mineral salt-producing reagent sources includes a sulfur supply from gaseous sulfur dioxide and/or dihydrogen sulfide. The systems and methods described herein can be used to produce the nitrogen containing mineral salt. The nitrogen containing mineral salt may be any of those recited herein, such as listed in the tables, and may include sulfates, nitrates, and phosphates. Micronutrients may also be included.

In one embodiment, the nitrate mineral can be formed to be devoid of one or more of the following elements or substances: Hg, U, B, Pb, Ni, Sb, As, Bi, Co, CN, Mo, Sn, V, or combinations thereof, or devoid of Hg, U, B, Pb, Sb, As, Bi, Mo, or combinations thereof. The nitrate mineral can be any of those listed devoid of these components.

In one embodiment, the nitrate mineral can be formed to include one or more of the following elements or substances: Ca, Fe, Ni, Al, C, H, Mn, SO4, PO4, K, H2, Sb, As, Cu, Mg, Si, F, Cl, Co, CN, Mo, Sn, V, CH3, or combinations thereof; or Ca, Fe, Al, C, H, Mn, SO4, PO4, K, H2, Cu, Mg, Si, F, Cl, CH3, or combinations thereof; or Ca, Fe, Al, C, H, Mn, SO4, PO4, K, H2, Mg, or combinations thereof. The nitrate mineral can be any of those listed with these components.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

As used herein, the term “organic” is meant to refer to the organic agricultural designation, which is a production system that sustains the health of soils, ecosystems and people. It relies on ecological processes, biodiversity and cycles adapted to local conditions, rather than the use of inputs with adverse effects. Unless clearly indicated, “organic” is not meant to refer to organic chemistry or organic substances defined by having a carbon. While the reagents or products may use a carbon, the recitation of being organic is in reference to the organic agricultural designation. As such, the organic acids or organic fertilizers or organic mineral salts may be referred to as merely acids or merely fertilizers or merely mineral salts.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

The following references are incorporated herein and describe microbes that can be used as described herein for producing or leading to the production of sulfuric acid: Muyzer, G. and Stams, A. J. (June 2008). “The ecology and biotechnology of sulfate-reducing bacteria” (PDF). Nature Reviews Microbiology 6: 441-454. doi:10.1038/nrmicro1892.PMID 18461075; Ernst-Detlef Schulze, Harold A. Mooney (1993), Biodiversity and ecosystem function, Springer-Verlag, pp. 88-90; Barton, Larry L. and Fauque, Guy D. (2009). “Biochemistry, Physiology and Biotechnology of Sulfate-Reducing Bacteria”. Advances in Applied Microbiology 68: 41-98. doi:10.1016/s0065-2164(09)01202-7; Larry Barton (ed.) (1995), Sulfate-reducing bacteria, Springer; Kasper U. Kjeldsen, Catherine Joulian, and Kjeld Ingvorsen (2004). “Oxygen Tolerance of Sulfate-Reducing Bacteria in Activated Sludge”. Environmental Science and Technology 38 (7): 2038-2043. doi:10.1021/es034777e.; Dexter Dyer, Betsey (2003). A Field Guide to Bacteria. Comstock Publishing Associates/Cornell University Press; Peter D. Ward (October 2006), “Impact from the Deep”, Scientific American; and Pfennig N. and Biebel H. (1986), “The dissimilatory sulfate-reducing bacteria”, in Starr et al., The Prokaryotes: a handbook on habitats, isolation and identification of bacteria, Springer.

1. A method of producing sulfuric acid, the method comprising:
obtaining a microbial culture that produces sulfuric acid;
placing the microbial culture in a bioreactor in an aqueous environment;
introducing an aqueous, liquid or gaseous sulfur supply into the bioreactor; and
culturing the microbial culture with the sulfur supply sufficiently so that sulfuric acid is produced.

2. The method of claim 1, wherein the sulfur supply is from gaseous sulfur dioxide and/or dihydrogen sulfide.

3. The method of claim 1, wherein the sulfur supply is aqueous sulfur dioxide and/or dihydrogen sulfide.

4. The method of claim 1, wherein the sulfur supply is sulfurous acid.

5. The method of claim 1, wherein the produced sulfuric acid is microbially produced sulfuric acid (MPSA) from an organic sulfur supply.

6. The method of claim 1, wherein the microbes are any microbes that processes sulfur so that sulfuric acid is produced.

7. The method of claim 1, wherein the microbes are any natural microbes that processes sulfur so that sulfuric acid is produced.

8. The method of claim 1, wherein the microbes are any genetically modified microbes that processes sulfur so that sulfuric acid is produced.

9. The method of claim 1, wherein the microbes are any cultivated microbes that processes sulfur so that sulfuric acid is produced.

10. The method of claim 1, wherein the microbes are any purchased microbes that processes sulfur so that sulfuric acid is produced.

11. The method of claim 6, wherein the microbes include other types of microbes that facilitate culturing of the sulfuric acid producing microbes.

12. The method of claim 1, comprising:
burning sulfur to obtain gaseous sulfur dioxide; and
bubbling gaseous sulfur dioxide through the aqueous environment of the bioreactor having the microbes.

13. The method of claim 1, comprising:
burning sulfur to obtain gaseous sulfur dioxide;
mixing the sulfur dioxide with water to form aqueous sulfur dioxide; and
injecting aqueous sulfur dioxide into the aqueous environment of the bioreactor having the microbes.

14. The method of claim 1, comprising:
burning sulfur to obtain gaseous sulfur dioxide;
mixing the sulfur dioxide with water to form sulfurous acid; and
injecting sulfurous acid into the aqueous environment of the bioreactor having the microbes.

15. The method of claim 1, wherein the bioreactor is part of a sulfurous acid producing system.

16. The method of claim 1, wherein the bioreactor receives sulfurous acid from a sulfurous acid producing system.

17. The method of claim 1, wherein the sulfuric acid is produced at ambient conditions.

18. The method of claim 1, wherein the microbes freely float or are suspended in the aqueous environment.

19. The method of claim 18, wherein the microbes are on a substrate that freely float or is suspended in the aqueous environment.

20. The method of claim 18, wherein the microbes are on a substrate affixed to an internal surface of the bioreactor.

21. The method of claim 1, wherein the process is a batch process.

22. The method of claim 1, wherein the process is a continuous process.

23. The method of claim 1, comprising:
recycling an aqueous liquid from the bioreactor through a sulfurous acid producing system until reaching a threshold pH, the aqueous liquid having a higher pH than the threshold; and
once the threshold pH is reached, extracting sulfuric acid from the bioreactor.

24. The method of claim 23, comprising:
measuring the pH of the aqueous environment; and
selectively extracting sulfuric acid when the pH is lower than the threshold pH; and
selectively recycling sulfurous acid when the pH is above the threshold pH.

25. The method of claim 1, comprising producing sulfuric acid until producing a composition that is at least 0.1%, 1% 10%, 29%, 32%, 62%, 70%, 78%, 80%, or 98% sulfuric acid or any range between any of the values of sulfuric acid.

26. The method of claim 1, comprising:
anaerobically digesting animal waste to produce digestate; and
providing the digestate to the bioreactor.

27. The method of claim 1, comprising:
anaerobically digesting animal waste to produce digestate gas; and
providing the digestate gas to the bioreactor.

28. The method of claim 1, comprising culturing the microbes that produce sulfuric acid by:
locating a geothermal area;
obtaining a microbial culture from the geothermal area;
selecting microbes from the microbial culture that produce sulfuric acid; and
culturing the selected microbes with an aqueous, liquid or gaseous sulfur supply.

29. The method of claim 28, wherein the geothermal area includes a fumarole, hot pot, warm spring, geyser, hot vent, volcanic formation, or any other geological formation.

30. The method of claim 1, comprising culturing the microbes that produce sulfuric acid by:
locating geologic area that is contaminated and has acidic water;
obtaining a microbial culture from the contaminated geologic area;
selecting microbes from the microbial culture that produce sulfuric acid; and
culturing the selected microbes with an aqueous, liquid or gaseous sulfur supply.

31. The method of claim 30, wherein the contaminated geologic area includes a mine having mine waste water or mine tailings or mineral processing plant or waste stream or tailings or dump or anywhere there is a sulfur contamination.

32. A method of producing a sulfate mineral salt, the method comprising:
culturing the microbial culture with the sulfur supply sufficiently so that the sulfuric acid is produced as in claim 1;
introducing a mineral salt-producing reagent source into the bioreactor and
culturing the microbial culture with the sulfur supply and mineral salt-producing reagent source sufficiently so that the sulfuric acid is produced and reacts with the mineral salt-producing reagent so as to produce the sulfate mineral salt.

33. The method of claim 32, wherein the sulfur supply is from gaseous sulfur dioxide.

34. The method of claim 32, wherein the sulfur supply is from solid sulfur.

35. The method of claim 32, wherein the sulfur supply is from particulate sulfur.

36. The method of claim 32, wherein the sulfur supply is from particulate sulfur having a dimension less than 37 microns.

37. The method of claim 32, wherein the sulfur supply is from particulate sulfur having a dimension less than 1 micron.

38. The method of claim 32, wherein the sulfur supply is from a sulfur mineral.

39. The method of claim 32, wherein the sulfur supply is from a sulfide.

40. The method of claim 32, wherein the sulfur supply is from pyrite.

41. The method of claim 32, wherein the sulfur supply is aqueous sulfur dioxide.

42. The method of claim 32, wherein the sulfur supply is sulfurous acid.

43. The method of claim 32, wherein the produced sulfuric acid is microbially produced sulfuric acid (MPSA) from an organic sulfur supply, the mineral salt-producing reagent is from an organic supply, and the resulting fertilizer is organic.

44. The method of claim 32, wherein the microbes are any microbes that process sulfur.

45. The method of claim 44, wherein the microbes include other type of microbes that facilitate culturing of the sulfuric acid producing microbes.

46. The method of claim 32, comprising:
burning sulfur to obtain gaseous sulfur dioxide; and
bubbling gaseous sulfur dioxide through the aqueous environment of the bioreactor having the microbes.

47. The method of claim 32, comprising:
burning sulfur to obtain gaseous sulfur dioxide;
mixing the sulfur dioxide with water to form aqueous sulfur dioxide; and
injecting aqueous sulfur dioxide into the aqueous environment of the bioreactor having the microbes.

48. The method of claim 32, comprising:
burning sulfur to obtain gaseous sulfur dioxide;
mixing the sulfur dioxide with water to form sulfurous acid; and
injecting sulfurous acid into the aqueous environment of the bioreactor having the microbes.

49. The method of claim 32, wherein the bioreactor is part of a sulfurous acid producing system.

50. The method of claim 32, wherein the bioreactor receives sulfurous acid from a sulfurous acid producing system.

51. The method of claim 32, wherein the sulfuric acid and mineral salt are produced at ambient conditions.

52. The method of claim 32, wherein the microbes freely float or are suspended in the aqueous environment.

53. The method of claim 52, wherein the microbes are on a substrate that freely float or are suspended in the aqueous environment.

54. The method of claim 52, wherein the microbes are on a substrate affixed to an inner surface of the bioreactor.

55. The method of claim 32, wherein the process is a batch process.

56. The method of claim 32, wherein the process is a continuous process.

57. The method of claim 32, comprising:
recycling an aqueous liquid from the bioreactor through a sulfurous acid producing system until reaching a threshold pH, the aqueous liquid having a higher pH than the threshold; and
once the threshold pH is reached, extracting sulfuric acid from the bioreactor.

58. The method of claim 57, comprising:
measuring the pH of the aqueous environment; and
selectively extracting sulfuric acid when the pH is lower than the threshold pH; and
selectively recycling sulfurous acid when the pH is above the threshold pH.

59. The method of claim 32, comprising producing sulfuric acid until the composition is at least 0.1%, 1%, 10%, 29-32%, 62-70%, 78-80%, or 98% sulfuric acid.

60. The method of claim 32, wherein the mineral salt-producing reagent source is organic.

61. The method of claim 60, wherein the mineral salt-producing reagent source is from excrement, compost, feces, urine or combination thereof.

62. The method of claim 32, wherein the mineral salt-producing reagent source is from a Berkley Technique composting silo.

63. The method of claim 32, comprising collecting the mineral salt as a precipitate.

64. The method of claim 32, comprising:
withdrawing liquid from the bioreactor; and
processing the liquid to obtain the sulfate mineral salt.

65. The method of claim 32, comprising:
withdrawing liquid from the bioreactor; and
centrifuging the liquid to obtain the sulfate mineral salt.

66. The method of claim 65, comprising recycling liquid from the centrifuge back to the bioreactor.

67. The method of claim 32, comprising making the smell of a farm better by making the sulfate mineral salt from animal waste.

68. The method of claim 32, comprising using equipment to remove water from the sulfate mineral salt.

69. The method of claim 32, comprising using equipment to remove the sulfate mineral salt from the aqueous environment.

70. The method of one of claims 32-69, wherein the mineral salt is ammonium sulfate ((NH₄)₂SO₄), Arcanite (K₂SO₄), Langbeinite, Anhydrite/Gypsum (CaSO₄), and Epsomite (Epsom Salts) (MgSO₄).

71. The method of one of claims 32-69, wherein the mineral salt is a sulfate, nitrate, and/or phosphate.

72. The method of one of the claims 32-69, wherein the mineral salt is produced using microbially produced sulfuric acid (MPSA).

73. The method of one of clams 32-69, wherein the mineral salt is any mineral salt that include phosphates, nitrates, and sulfates.

74. The method of one of claims 32-69, wherein the mineral salt is ammonium sulfate, potassium sulfate, potassium magnesium sulfate, gypsum (e.g., calcium sulfate), sulfur-containing nitrogen or nitrogen phosphorus potassium (NPK) salts.

75. The method of one of claims 32-69, wherein the mineral salt is produced with the MPSA and includes nitrogen, phosphorus, or potassium; nitrogen-sulfur such as ammonium sulfate, ammonium nitrate-sulfate, ammonium phosphate-sulfate, and ammonium phosphate-nitrate; and potassium-sulfur mineral salts such as potassium sulfate and potassium magnesium-sulfate.

76. The method of one of claims 32-69, wherein the mineral salt includes micronutrients such as ZnSO₄ and FeSO_{4,} or others.

77. The method of claim 1 or claim 32, comprising:
anaerobically digesting animal waste to produce digestate; and
providing the digestate to the bioreactor.

78. The method of claim 1 or claim 32, comprising:
anaerobically digesting animal waste to produce digestate gas; and
providing the digestate gas to the bioreactor.

79. The method of claim 32, comprising culturing microbes that produce sulfuric acid by:
locating a geothermal area;
obtaining a microbial culture from the geothermal area;
selecting microbes from the microbial culture that produce sulfuric acid; and
culturing the selected microbes with an aqueous, liquid or gaseous sulfur supply.

80. The method of claim 79, wherein the geothermal area includes a fumarole, hot pot, warm spring, geyser, hot vent, volcanic formation, or any other geological formation.

81. The method of claim 32, comprising culturing microbes that produce sulfuric acid by:
locating geologic area that is contaminated and has acidic water;
obtaining a microbial culture from the contaminated geologic area;
selecting microbes from the microbial culture that produce sulfuric acid; and
culturing the selected microbes with an aqueous, liquid or gaseous sulfur supply.

82. The method of claim 81, wherein the contaminated geologic area includes a mine having mine waste water or mine tailings or mineral processing plant or waste stream or tailings or dump or anywhere there is a sulfur contamination.

83. The method of one of the claims of claims 32-69, comprising:
introducing a phosphate to the MPSA; and
producing a phosphate mineral salt.

84. The method of one of the claims of claims 32-69, comprising:
introducing a rock or milled particle thereof having phosphate or derivative there to the MPSA; and
producing a phosphate mineral salt.

85. The method of one of the claims of claims 32-69, comprising:
introducing a phosphate to the MPSA to produce phosphoric acid; and
producing a phosphate mineral salt from the phosphoric acid and counter ions from reagents in the bioreactor.

86. The method of one of the claims of claims 32-69, comprising:
introducing a phosphate to the MPSA to produce phosphoric acid.

87. The method of one of the claims of claims 32-69, comprising:
introducing a carbonic acid or carbonate to the MPSA; and
producing a carbonate mineral salt.
